(19) United States
(12) Patent Application Publication
(10) Pub. No.: US 2024/0366814 A1
(43) Pub. Date: Nov. 7, 2024

(54) TREATMENT OF BLADDER CANCER WITH RADIOLABELED ANTIBODIES TARGETING CDCP1

(71) Applicant: The Regents of the University of California, Oakland, CA (US)

(72) Inventors: Michael J. EVANS, Pacifica, CA (US); James A. WELLS, Burlingame, CA (US); Jonathan CHOU, San Francisco, CA (US)

(73) Assignee: THE REGENTS OF THE UNIVERSITY OF CALIFORNIA, Oakland, CA (US)

(21) Appl. No.: 18/655,081

(22) Filed: May 3, 2024

Related U.S. Application Data
(60) Provisional application No. 63/500,528, filed on May 5, 2023.

(51) Int. Cl. A61K 51/10; A61K 9/00; A61K 39/00; A61K 47/68; A61P 35/00; C07K 16/28

(52) U.S. Cl. CPC A61K 51/1027; A61K 9/0019; A61K 47/6803; A61P 35/00; C07K 16/2896; A61K 2039/505; C07K 2317/565

(57) ABSTRACT

The disclosure relates to radioimmunotherapy for bladder cancer using an antibody or antibody fragment that specifically binds CUB domain-containing protein 1 (CDCP1). The disclosure also relates to use of a diagnostic radioisotope-labeled anti-CDCP1 antibody or CDCP1-binding fragment thereof for radionuclide imaging of CDCP1 expression in vivo in bladder cancer patients.

### CROSS-REFERENCE TO RELATED APPLICATION(S)

This application claims priority to and benefit of U.S. Provisional Patent Application No. 63/500,528, filed May 5, 2023, the content of which is herein incorporated by reference in its entirety.

### STATEMENT REGARDING FEDERALLY SPONSORED RESEARCH OR DEVELOPMENT

This invention was made with government support under W81XWH-21-1-0498 awarded by the Medical Research and Development Command. The government has certain rights in the invention.

### REFERENCE TO AN ELECTRONIC SEQUENCE LISTING

The content of the electronic sequence listing (643662003000SEQLIST.xml; Size: 73,084 bytes; and Date of Creation: Apr. 17, 2024) is herein incorporated by reference in its entirety.

### FIELD

The disclosure relates to radioimmunotherapy for bladder cancer using an antibody or antibody fragment that specifically binds CUB domain-containing protein 1 (CDCP1). The disclosure also relates to use of a diagnostic radioisotope-labeled anti-CDCP1 antibody or CDCP1-binding fragment thereof for radionuclide imaging of CDCP1 expression in vivo in bladder cancer patients.

### BACKGROUND

Bladder cancer (BC) is the second most common malignancy of the genitourinary tract, leading to over 17,000 deaths in the United States each year (Lenis et al., JAMA, 324(10):1980-1991, 2020). Despite advances in imaging, chemotherapy, and surgery, the survival of patients who develop metastatic bladder cancer (mBC) remains poor. Treatments for mBC were historically limited to platinum-based chemotherapy, until the approval of the first checkpoint inhibitor (Rosenberg et al., Lancet, 387(10031):1909-1920, 2016). However, anti-PD1 therapy is only effective in about 25% of patients (Tran et al., Nat Rev Cancer, 21(2):104-121, 2021). More recently, two antibody drug conjugates (ADCs), enfortumab vedotin (which targets the surface protein NECTIN4) and sacituzumab govitecan (which targets the surface protein TROP2) received full and accelerated Food and Drug Administration (FDA) approval, respectively, based on trials conducted in heavily pre-treated patients (Rosenberg et al., J Clin Oncol, 37(29):2592-2600, 2019; Tagawa et al., J Clin Oncol, 39(22):2474-2485, 2021; and Powles et al., N Engl J Med, 384(12):1125-1135, 2021). With the milestone FDA approvals, these therapies are now being tested in earlier disease settings.

Muscle-invasive bladder cancer (MIBC) is a molecularly diverse disease and has been classified into six molecular subtypes (Choi et al., Cancer Cell, 25(2):152-165, 2014). As such, not all subtypes of MIBC are likely to express sufficient TROP2 or NECTIN4 to be vulnerable to the approved cognate ADCs (Chou et al., Eur Urol Oncol, 2022; and Kamoun et al., Eur Urol, 77(4):420-433, 2020). Recently, NECTIN4 was found to be highly enriched in the luminal subtypes of MIBC, which is critical for drug response, with comparatively lower expression in the four other subtypes (Chu et al., Clin Cancer Res, 27(18):5123-5130, 2021). These data suggest a molecular diversity to MIBC that will undoubtedly necessitate a large repertoire of therapeutics targeting other antigens expressed in NECTIN4 or TROP2 null tumors.

Thus, the identification of additional cell surface antigens as targets for treatment of bladder cancer are needed, particularly for bladder cancer subtypes that are resistant to currently approved therapeutics.

### SUMMARY

The disclosure relates to radioimmunotherapy for bladder cancer using an antibody or antibody fragment that specifically binds CUB domain-containing protein 1 (CDCP1). The disclosure also relates to use of a diagnostic radioisotope-labeled anti-CDCP1 antibody or CDCP1-binding fragment thereof for radionuclide imaging of CDCP1 expression in vivo in bladder cancer patients.

### BRIEF DESCRIPTION OF THE DRAWINGS

FIG. 1A-1E illustrate CDCP1 expression across the consensus molecular subtypes of muscle-invasive bladder cancer (MIBC). FIG. 1A shows the expression levels of CDCP1 in six molecular subtypes of bladder cancer in the Seiler 2017 dataset. FIG. 1B shows the expression levels of CDCP1 in six molecular subtypes of bladder cancer in the Sjödahl 2017 dataset. FIG. 1C shows the expression levels of CDCP1 in six molecular subtypes of bladder cancer in the TCGA dataset. FIG. 1D shows the expression levels of CDCP1 in six molecular subtypes of bladder cancer in the Sjödahl 2012 dataset. FIG. 1E shows CDCP1 protein was enriched in the basal/squamous (Ba/Sq) subtype.

FIG. 2A-2D show CDCP1 protein expression in human bladder cancer samples. FIG. 2A shows CDCP1 protein expression on the cell surface in all human bladder cancer cell lines studied using flow cytometry. FIG. 2B shows protein expression of full length (140 kDa) CDCP1 and cleaved (70 kDa) CDCP1 in human bladder cancer cell lines and patient derived xenografts using immunoblot. The TROP2 and Nectin-4 immunoblot highlights the discordance with CDCP1 expression. FIG. 2C shows expression of CDCP1 in the TCGA cohorts is not correlated with expression of TROP2. FIG. 2D shows expression of CDCP1 in the TCGA cohorts is inversely correlated with expression of NECTIN4. The Spearman rank correlation coefficient and p-values are shown for each analysis.

FIG. 3A-3B show that a radiolabeled anti-CDCP1 antibody and positron emission tomography (PET) detects molecularly diverse human bladder cancer xenografts with varying levels of CDCP1 expression. FIG. 3A shows ex vivo tumor uptake data acquired 72 hours post injection of ⁸⁹Zr-4A06 (n=5 mice/tumor). FIG. 3B shows the tumor to muscle ratios calculated from postmortem biodistribution data collected 72 hours post injection of ⁸⁹Zr-4A06.

FIG. 4A-4D show that treatment with a radiolabeled anti-CDCP1 antibody suppresses bladder cancer growth. FIG. 4A shows growth curves collected from mice bearing subcutaneous UMUC3 xenografts post treatment with vehicle or a fractionated dose of ¹⁷⁷Lu-4A06 (2 doses of 400 μCi/dose, day 0 and day 7, n=9 mice/arm). The mean tumor volume was significantly smaller in the treated versus vehicle group by day 20 of the study. FIG. 4B is a Kaplan-Meier curve showing relative survival in the vehicle versus treated arms. ¹⁷⁷Lu-4A06 significantly extended survival compared to control. P<0.01. Four of nine mice in the treated arm experienced complete tumor regressions and no regrowth was documented out to 120 days post injection. FIG. 4C shows that treatment with ¹⁷⁷Lu-4A06 significantly suppressed tumor growth as compared to a vehicle control. The plot shows the mean tumor volume for the vehicle and treated arms from the antitumor assessment study in mice bearing UMUC3 xenografts. The differences in mean between treatment arms was found to be significant (P<0.001) using an unpaired, two tailed t test. Data are reported as mean±standard deviation. FIG. 4D shows mouse body weights from the antitumor assessment study. Data are reported as mean±standard deviation.

FIG. 5A-5F shows that CDCP1 expression is regulated by the RAS-Raf-MEK signaling pathway in bladder cancer. FIG. 5A is a schematic illustrating the RAS-Raf-MEK pathway downstream of receptor tyrosine kinase (RTK) signaling. The prevalence of molecular alterations (including mutations, structural variants and copy number alterations) in bladder cancer from cbioportal is indicated. FIG. 5B shows the prevalence of the most common RAS mutations in bladder cancer from cbioportal. FIG. 5C shows total CDCP1 expression in UMUC3 cells (which harbor a KRAS G12C mutation) treated with increasing concentrations of sotorasib, a KRAS G12C inhibitor, as determined by western blot. FIG. 5D shows that treatment of UMUC3 cells with sotorasib suppressed CDCP1 expression as determined using flow cytometry. FIG. 5E shows total CDCP1 expression in UMUC3 cells treated with increasing concentrations of trametinib, a MEK inhibitor, as determined by western blot. FIG. 5F shows that treatment of UMUC3 cells with trametinib suppressed CDCP1 expression as determined using flow cytometry.

### DETAILED DESCRIPTION

Despite the growing interest in the cell surface protein CUB domain containing protein 1 (CDCP1), very little is known about its expression in metastatic bladder cancer (mBC). This knowledge gap, as well as the potential upside of discovering a new cell surface target to treat BC led us to investigate whether: (1) CDCP1 is overexpressed in BC and in which subtypes, (2) CDCP1 overexpression occurs in TROP2 and/or NECTIN4 null BC tumors, and (3) CDCP1 can be exploited for targeted radiotherapy (TRT) to treat BC.

### Experimental Design

As described in Example 1, CDCP1 expression was evaluated in four bladder cancer datasets (n=1,047 biopsies). A tissue microarray of primary bladder cancer biopsies was probed for CDCP1 by immunohistochemistry (IHC). CDCP1 expression was evaluated in patient-derived xenografts and cell lysates by immunoblot, flow cytometry, and saturation binding assays. Tumor detection in mouse bladder cancer models was tested using ⁸⁹Zr-labeled 4A06, a monoclonal antibody targeting the ectodomain of CDCP1. ¹⁷⁷Lu-4A06 was applied to mice bearing UMUC3 or HT-1376 xenografts to evaluate antitumor effects (CDCP1 expression in UMUC3 is 10-fold higher than HT-1376).

CDCP1 expression was highest in the basal/squamous subtype, and CDCP1 was expressed in 53% of primary biopsies. CDCP1 was not correlated with pathologic or tumor stage, metastatic site, or NECTIN4 and TROP2 at the mRNA or protein level. CDCP1 ranged from 10⁵ to 10⁶ receptors per cell. Mechanism studies showed that RAS signaling induced CDCP1 expression. ⁸⁹Zr-4A06 PET detected five human bladder cancer xenografts. ¹⁷⁷Lu-4A06 inhibited the growth of UMUC3 and HT-1376 xenografts, models with high and moderate CDCP1 expression, respectively. These data establish that CDCP1 is expressed in bladder cancer, including TROP2 and NECTIN4-null disease, and demonstrate that bladder cancer can be treated with CDCP1-targeted radiotherapy.

The disclosure relates to radioimmunotherapy for bladder cancer using an antibody or antibody fragment that specifically binds CUB domain-containing protein 1 (CDCP1). The disclosure also relates to use of a diagnostic radioisotope-labeled anti-CDCP1 antibody or CDCP1-binding fragment thereof for radionuclide imaging of CDCP1 expression in vivo in bladder cancer patients.

### Definitions

As used herein and in the appended claims, the singular forms “a,” “an,” and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments. It is understood that aspects and embodiments described herein as “comprising” include “consisting of” and “consisting essentially of” embodiments.

The term “about” as used herein in reference to a value, encompasses from 90% to 110% of that value (e.g., about 200 mCi radiolabeled antibody refers to 180 mCi to 220 mCi radiolabeled antibody and includes 200 mCi radiolabeled antibody).

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats). In some preferred embodiments, the subject is a human bladder cancer patient.

### 1. Anti-CDCP1 Antibodies

In one aspect, provided herein are antibodies and antibody fragments that specifically bind the CUB-domain containing protein 1 (CDCP1). The CDCP1 protein, which is also referred to in the literature as SIMA135, CD318 and TRASK (transmembrane and associated with src kinases), has a large extracellular domain of 665 amino acids and contains three CUB domains. The CUB domains mediate protein-protein interactions and are presumed to be involved in cell adhesion and interaction with the extracellular matrix (Bhatt et al., Oncogene, 24:5333-5343, 2005; and Casar et al., Oncogene, 33:255-268, 2014). For example, CDCP1 cellular adhesion plays a role in controlling phosphorylation status in at least keratinocytes. As described herein, CDCP1 expression appears to be regulated by RAS.

As used herein, CUB domain-containing protein 1 (CDCP1) is meant to include the extracellular portion of the full length CDCP1 protein. As is well known in the art, CDCP1 is a glycosylated transmembrane protein with an extracellular domain of approximately 638 amino acids. The full-length human version of CDCP1, after cleaving of the signal peptide (residues 1-29 of the full-length chain) is 807 amino acids in length. The amino acid sequence of human CDCP1 is found at Uniprot Record No. Q9H5V8, the entirely of which is incorporated by reference. As used herein, CDCP1 includes the human version and any natural variant thereof. For example, one natural variant of CDCP1 includes a Q525R mutation (with the numbering including the first 29 amino acids as the signal sequence that is subsequently cleaved), which is in the extracellular domain of the CDCP1. The antibody or antibody fragments of the present disclosure bind to the extracellular portion of the “mature” (lacking signal peptide) version of the CDCP1 protein.

As used herein, the term “antibody” refers to an immunoglobulin molecule that can specifically bind to a particular antigen. Antibodies have different isotypes or classes, such as but not limited to the isotypes known as IgA, IgD, IgE, IgG and IgM. The term antibody as used herein encompasses all isotypes. As is well understood in the art, a typical antibody is composed of two identical heavy chains and two identical light chains. The heavy chains are joined to one another via at least one disulfide bond and each light chain is joined to a heavy chain via a disulfide bond. Each heavy and light chain generally comprises a “variable domain” (VH and VL, respectively) at or near the N-terminus of the antibody. The variable domains for each chain are critical for antigen binding. The light chain contains one additional “constant region” (CL), and the heavy chain contains three or four additional constant regions (CH1, CH2, CH3, CH4). Thus, in specific embodiments, any of the antibodies provided herein may be IgA antibodies, IgD antibodies, IgE antibodies, IgG antibodies, or IgM antibodies. In other specific embodiments, any of the antibodies provided herein is a monoclonal antibody, e.g., comprising two identical light chains and two identical heavy chains.

The VH and VL chains generally each comprise three complementarity determining regions (CDRs) that determine antigen binding specificity. The CDRs can also be referred to as “loops” or “L regions.” The framework regions (FRs) are amino acid stretches within the VH and VL intervening between the CDRs. In full length VH and VL chains, each chain comprises three CDRs (Ls) and four framework regions (FR1 through FR4). The 4 FRs are separated respectively by the three CDRs (CDR1, CDR2, CDR3) or (L1, L2, L3). The CDRs, and in particular the CDR3 regions, and more particularly the VH CDR3, may, in certain embodiments, be largely responsible for antibody specificity.

A Fab fragment (fragment antigen-binding) is a region of an antibody that binds to antigens. Fab fragments may comprise one constant domain (CL and CH1) and one variable chain (VL and VH) of each of the heavy and the light chain. F(ab′)₂ refers to an antibody fragment comprising a Fab dimer. Fab and F(ab′)₂ may be generated by recombinant technology or by cleavage of an antibody or a fragment of antibody.

In some embodiments, the present disclosure provides monoclonal antibodies, or antigen-binding fragments thereof, which specifically bind to human CDCP1. The monoclonal antibody may be a human antibody, a humanized antibody, or a chimeric antibody, and may include a constant region. In some embodiments, the human constant region is selected from the group consisting of IgG1, IgG2, IgG3 and IgG4 constants regions. In some embodiments, the antigen-binding fragment is selected from the group consisting of Fab, Fab′-SH, F(ab′)₂, scFV and Fv fragments.

An antibody fragment may comprise part of an immunoglobulin molecule or a combination of parts of immunoglobulin molecules. In specific embodiments, the antibody fragments provided herein retain the ability to bind the same antigen that the full-length antibody binds, e.g., CDCP1, e.g., a CUB domain of CDCP1. The fragment may or may not bind to the exact same epitope as the full-length antibody from which the fragment is derived. Antibody fragments include but are not limited to F(ab′)₂, Fab, Fv and Fc fragment, as well as fusion peptide such as single chain Fv (scFv) fragments. ScFv fragments are single chain peptides that contain a VH chain and the VL chain, e.g., any of the VH and VL chains provided herein, linked to one another via a linker peptide. In specific embodiments, the connector peptide ranges from about two to about 50 amino acids. In some embodiments, the connector peptide ranges from about two to about 10 amino acids, from about 10 to about 15 amino acids, from about 15 to about 20 amino acids, from about 20 to about 25 amino acids, from about 25 to about 30 amino acids, from about 30 to about 35 amino acids, from about 35 to about 40 amino acids, from about 40 to about 45 amino acids or from about 45 to about 50 amino acids. The ScFv may retain the antigen binding ability of the original immunoglobulin molecule.

In select embodiments, the antibodies or antibody fragments provided herein comprise the variable heavy chain (VH) amino acid sequence of the Fab heavy chain of SEQ ID NO:1:

EISEVQLVES GGGLVQPGGS LRLSCAASGF NFSSSSIHWV RQAPGKGLEW VASISSSYGY
TYYADSVKGR FTISADTSKN TAYLQMNSLR AEDTAVYYCA RTVRGSKKPY FSGWAMDYWG
QGTLVTVSSA STKGPSVFPL APSSKSTSGG TAALGCLVKD YFPEPVTVSW NSGALTSGVH
TFPAVLQSSG LYSLSSVVTV PSSSLGTQTY ICNVNHKPSN TKVDKKVEPK SCDKTHTGGS
HHHHHH.

In other embodiments, the antibodies or antibody fragments of the present disclosure comprise the variable light chain (VL) amino acid sequence of the Fab heavy chain of SEQ ID NO:9:

SDIQMTQSPS SLSASVGDRV TITCRASQSV SSAVAWYQQK PGKAPKLLIY SASSLYSGVP
SRESGSRSGT DETLTISSLQ PEDFATYYCQ QSSYSLITFG QGTKVEIKRT VAAPSVFIFP
PSDSQLKSGT ASVVCLLNNF YPREAKVQWK VDNALQSGNS QESVTEQDSK DSTYSLSSTL
TLSKADYEKH KVYACEVTHQ GLSSPVTKSF NRGECGGSDY KDDDDK.

In still other embodiments, the antibodies or antibody fragments comprise both the VH of SEQ ID NO:1 and the VL of SEQ ID NO:9. The antibody or antibody fragment (i.e., CDCP1-binding fragment) containing the VH and VL of SEQ ID NO:1 and SEQ ID NO:9, respectively, is referred to herein as CDCP1-001. As used herein, the term “antibody fragment” refers to an “antigen-binding fragment” (i.e., CDCP1-binding fragment) of the designated antibody.

The underlined portions of the VH and VL amino acid sequences of the Fab heavy and light chains above represent the complementarity determining regions (CDRs) in each chain, as per the IMGT system (Lefranc et al., Dev Comp Immunol, 27:55-77, 2003). For example, CDR1 of the VH is the amino acid sequence FSSSSI (SEQ ID NO: 3), CDR2 of the VH is the amino acid sequence SISSSYGYTY (SEQ ID NO: 5) and CDR3 of the VH is the amino acid sequence is TVRGSKKPYFSGWAM (SEQ ID NO: 7). The non-underlined portions of the sequences above represent the framework regions (FRs). For example, FR1 of the VH is the amino acid sequence EISEVOLVESGGGLVQPGGSLRLSCAASGEN (SEQ ID NO:2), FR2 of the VH is the amino acid sequence HWVRQAPGKGLEWVA (SEQ ID NO:4), FR3 of the VH is the amino acid sequence YADSVKGRFTISADTSKNTAYLQMNSLRAEDTAVYYCAR (SEQ ID NO:6) and FR4 of the VH is the amino acid sequence DYWGQGTLVTVSS (SEQ ID NO:8). Likewise, CDR1 of VL is the amino acid sequence RASQSVSSAVA (SEQ ID NO:11), CDR2 of VL is the amino acid sequence SASSLYS (SEQ ID NO:13) and CDR3 of the VL is the amino acid sequence SSYSLI (SEQ ID NO:15). The FR1 of the VL is the amino acid sequence SDIQMTQSPSSLSASVGDRVTITC (SEQ ID NO:10), FR2 of VL is the amino acid sequence WYQQKPGKAPKLLIY (SEQ ID NO:12), FR3 of VL is the amino acid sequence GVPSRFSGSRSGTDFTLTISSLQPEDFATYYCQQ (SEQ ID NO:14) and FR4 of the VL is the amino acid sequence TFGQGTKVEIK (SEQ ID NO:16). In the exemplary Fab heavy and light chain sequences shown above, DKTHTGGSHHHHHH (SEQ ID NO:79) and GGSDYKDDDDK (SEQ ID NO:80) are affinity tags.

In other embodiments, the antibodies or antibody fragments comprise VH FR1-FR4, VL FR1-FR4 and VL CDR1-CDR2 of the CDCP1-001 fragment disclosed above, and different VH CDR1-CDR3 and VL CDR3, as shown below in Table A-1. In another embodiment, the antibodies or antibody fragments comprise VL CDR1-CDR2 of the CDCP1-001 fragment, and different VH CDR1-CDR3 VL CDR3, as shown below in Table A-1. In other words, in these specific embodiments, the antibodies or antibody fragments CDCP1-002 through CDCP1-011 would possess the VH CDRs and the VL CDR3 listed in Table A-1, respectively, plus the VL CDR1 (SEQ ID NO:11) and VL CDR2 (SEQ ID NO:13) from the CDCP1-001 fragment. For example, in one embodiment, the VH CDCP1-002 comprises the amino acid sequence from N-terminus to C-terminus: SEQ ID NO:2 (VH FR1), SEQ ID NO: 17 (VH CDR1), SEQ ID NO:4 (VH FR2), SEQ ID NO:18 (VH CDR2), SEQ ID NO:6 (VH FR3), SEQ ID NO: 19 (VH CDR3) and SEQ ID NO:8 (VH FR4). Similarly, in one embodiment, the VL CDCP1-002 comprises the amino acid sequence from N-terminus to C-terminus: SEQ ID NO: 10 (VL FR1), SEQ ID NO: 11 (VH CDR1), SEQ ID NO:12 (VH FR2), SEQ ID NO:13 (VH CDR2), SEQ ID NO:14 (VH FR3), SEQ ID NO:20 (VH CDR3) and SEQ ID NO:16 (VH FR4). The combination of VH FRs1-4 and VH CDRs1-3, in proper order, make up the VH chain for each of the 001-011 anti-CDCP1 antibody fragments. Similarly, the combination of VL FRs1-4 and VL CDRs1-3, in proper order, make up the VL chain for each of the 001-011 anti-CDCP1 antibody fragments.

TABLE A-1
Sequences of CDRs of Anti-CDCP1 Fabs

| Fab Region | CDR1 | CDR2 | CDR3 |
|---|---|---|---|
| CDCP1-001 V_{H} | FSSSSI | SISSSYGYTY | TVRGSKKPYFSGWAM |
| | (SEQ ID NO: 3) | (SEQ ID NO: 5) | (SEQ ID NO: 7) |
| CDCP1-001 V_{L} | RASQSVSSAVA | SASSLYS | SSYSLI |
| | (SEQ ID NO: 11) | (SEQ ID NO: 13) | (SEQ ID NO: 15) |
| CDCP1-002 V_{H} | LSYYYI | SIYSSSSYTS | AYYGF |
| | (SEQ ID NO: 17) | (SEQ ID NO: 18) | (SEQ ID NO: 19) |
| CDCP1-002 V_{L} | RASQSVSSAVA | SASSLYS | SYYYYPI |
| | (SEQ ID NO: 11) | (SEQ ID NO: 13) | (SEQ ID NO: 20) |
| CDCP1-003 V_{H} | ISYYSM | SISPYSGYTS | GYYAL |
| | (SEQ ID NO: 21) | (SEQ ID NO: 22) | (SEQ ID NO: 23) |
| CDCP1-003 V_{L} | RASQSVSSAVA | SASSLYS | YYYFYPF |
| | (SEQ ID NO: 11) | (SEQ ID NO: 13) | (SEQ ID NO: 24) |
| CDCP1-004 V_{H} | ISYYYM | SIYSSYGYTS | VYYGF |
| | (SEQ ID NO: 25) | (SEQ ID NO: 26) | (SEQ ID NO: 27) |
| CDCP1-004 V_{L} | RASQSVSSAVA | SASSLYS | SYYVYPI |
| | (SEQ ID NO: 11) | (SEQ ID NO: 13) | (SEQ ID NO: 28) |
| CDCP1-005 V_{H} | ISYYYI | SIYPYYGSTY | AYYGF |
| | (SEQ ID NO: 29) | (SEQ ID NO: 30) | (SEQ ID NO: 31) |
| CDCP1-005 V_{L} | RASQSVSSAVA | SASSLYS | SYWSFPI |
| | (SEQ ID NO: 11) | (SEQ ID NO: 13) | (SEQ ID NO: 32) |
| CDCP1-006 V_{H} | LYYSYM | YISPYSGSTY | YSYSAL |
| | (SEQ ID NO: 33) | (SEQ ID NO: 34) | (SEQ ID NO: 35) |
| CDCP1-006 V_{L} | RASQSVSSAVA | SASSLYS | SSWHYHLF |
| | (SEQ ID NO: 11) | (SEQ ID NO: 13) | (SEQ ID NO: 36) |
| CDCP1-007 V_{H} | IYSYYI | SIYPYYGYTS | AYYGM |
| | (SEQ ID NO: 37) | (SEQ ID NO: 38) | (SEQ ID NO: 39) |
| CDCP1-007 V_{L} | RASQSVSSAVA | SASSLYS | SYFYWPI |
| | (SEQ ID NO: 11) | (SEQ ID NO: 13) | (SEQ ID NO: 40) |
| CDCP1-008 V_{H} | ISSYYM | SIYPYSGYTY | AYYAM |
| | (SEQ ID NO: 41) | (SEQ ID NO: 42) | (SEQ ID NO: 43) |
| CDCP1-008 V_{L} | RASQSVSSAVA | SASSLYS | SYYVYPI |
| | (SEQ ID NO: 11) | (SEQ ID NO: 13) | (SEQ ID NO: 44) |
| CDCP1-009 V_{H} | LYSYYI | SIYPYYSSTS | YYYAM |
| | (SEQ ID NO: 45) | (SEQ ID NO: 46) | (SEQ ID NO: 47) |
| CDCP1-009 V_{L} | RASQSVSSAVA | SASSLYS | GYAGSWHPI |
| | (SEQ ID NO: 11) | (SEQ ID NO: 13) | (SEQ ID NO: 48) |
| CDCP1-010 V_{H} | IYSYSM | SISPYYSYTS | AYYAL |
| | (SEQ ID NO: 49) | (SEQ ID NO: 50) | (SEQ ID NO: 51) |
| CDCP1-010 V_{L} | RASQSVSSAVA | SASSLYS | SYWYYPI |
| | (SEQ ID NO: 11) | (SEQ ID NO: 13) | (SEQ ID NO: 52) |
| CDCP1-011 V_{H} | ISYYYM | SIYSSSSYTS | SYYAM |
| | (SEQ ID NO: 53) | (SEQ ID NO: 54) | (SEQ ID NO: 55) |
| CDCP1-011 V_{L} | RASQSVSSAVA | SASSLYS | SYYVYPI |
| | (SEQ ID NO: 11) | (SEQ ID NO: 13) | (SEQ ID NO: 56) |

TABLE A-2
Sequences of Variable Regions of Anti-CDCP1 Fabs^{∧}
Fab Variable Region
CDCP1-002 V_{H} (SEQ ID NO: 57)
EVQLVES GGGLVQPGGS LRLSCAASGF NLSYYYIHWV RQAPGKGLEW VASIYSSSSY
TSYADSVKGR FTISADTSKN TAYLQMNSLR AEDTAVYYCA RAYYGFDYWG QGTLVTVSS
CDCP1-002 V_{L} (SEQ ID NO: 58)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYYYYPITFG QGTKVEIK
CDCP1-003 V_{H} (SEQ ID NO: 59)
EVQLVES GGGLVQPGGS LRLSCAASGF NISYYSMHWV RQAPGKGLEW VASISPYSGY
TSYADSVKGR FTISADTSKN TAYLQMNSLR AEDTAVYYCA RGYYALDYWG QGTLVTVSS
CDCP1-003 V_{L} (SEQ ID NO: 60)
DIQMTQSPS SLSASVGDRV TITCRASQSV SSAVAWYQQK PGKAPKLLIY SASSLYSGVP
SRFSGSRSGT DFTLTISSLQ PEDFATYYCQ QYYYFYPFTFG QGTKVEIK
CDCP1-004 V_{H} (SEQ ID NO: 61)
EVQLVES GGGLVQPGGS LRLSCAASGF NISYYYMHWV RQAPGKGLEW VASIYSSYGY
TSYADSVKGR FTISADTSKN TAYLQMNSLR AEDTAVYYCA RVYYGFDYWG QGTLVTVSS
CDCP1-004 V_{L} (SEQ ID NO: 62)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYYVYPITFG QGTKVEIK
CDCP1-005 V_{H} (SEQ ID NO: 63)
EVQLVES GGGLVQPGGS LRLSCAASGF NISYYYIHWV RQAPGKGLEW VASIYPYYGS
TYYADSVKGR FTISADTSKN TAYLQMNSLR AEDTAVYYCA RAYYGFDYWG QGTLVTVSS
CDCP1-005 V_{L} (SEQ ID NO: 64)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYWSFPITFG QGTKVEIK
CDCP1-006 V_{H} (SEQ ID NO: 65)
EVQLVES GGGLVQPGGS LRLSCAASGF NLYYSYMHWV RQAPGKGLEW VAYISPYSGS
TYYADSVKGR FTISADTSKN TAYLQMNSLR AEDTAVYYCA RYSYSALDYWG QGTLVTVSS
CDCP1-006 V_{L} (SEQ ID NO: 66)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSSWHYHLFTFG QGTKVEIK
CDCP1-007 V_{H} (SEQ ID NO: 67)
EVQLVES GGGLVQPGGS LRLSCAASGF NIYSYYIHWV RQAPGKGLEW VASIYPYYGY
TSYADSVKGR FTISADTSKN TAYLQMNSLR AEDTAVYYCA RAYYGMDYWG QGTLVTVSS
Fab Variable Region
CDCP1-007 V_{L} (SEQ ID NO: 68)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYFYWPITFG QGTKVEIK
CDCP1-008 V_{H} (SEQ ID NO: 69)
EVQLVES GGGLVQPGGS LRLSCAASGF NISSYYMHWV RQAPGKGLEW VASIYPYSGY
TYYADSVKGR FTISADTSKN TAYLQMNSLR AEDTAVYYCA RAYYAMDYWG QGTLVTVSS
CDCP1-008 V_{L} (SEQ ID NO: 70)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYYVYPITFG QGTKVEIK
CDCP1-009 V_{H} (SEQ ID NO: 71)
EVQLVES GGGLVQPGGS LRLSCAASGF NLYSYYIHWV RQAPGKGLEW VASIYPYYSS
TSYADSVKGR FTISADTSKN TAYLQMNSLR AEDTAVYYCA RYYYAMDYWG QGTLVTVSS
CDCP1-009 V_{L} (SEQ ID NO: 72)
DIQMTQSPS SLSASVGDRV TITCRASQSV SSAVAWYQQK PGKAPKLLIY SASSLYSGVP
SRFSGSRSGT DFTLTISSLQ PEDFATYYCQ QGYAGSWHPITFG QGTKVEIK
CDCP1-010 V_{H} (SEQ ID NO: 73)
EVQLVES GGGLVQPGGS LRLSCAASGF NIYSYSMHWV RQAPGKGLEW VASISPYYSY
TSYADSVKGR FTISADTSKN TAYLQMNSLR AEDTAVYYCA RAYYALDYWG QGTLVTVSS
CDCP1-010 V_{L} (SEQ ID NO: 74)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYWYYPITFG QGTKVEIK
CDCP1-011 V_{H} (SEQ ID NO: 75)
EVQLVES GGGLVQPGGS LRLSCAASGF NISYYYMHWV RQAPGKGLEW VASIYSSSSY
TSYADSVKGR FTISADTSKN TAYLQMNSLR AEDTAVYYCA RSYYAMDYWG QGTLVTVSS
CDCP1-011 V_{L} (SEQ ID NO: 76)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSYYVYPITFG QGTKVEIK
CDCP1-001 V_{H} (SEQ ID NO: 77)
EVQLVES GGGLVQPGGS LRLSCAASGF NFSSSSIHWV RQAPGKGLEW VASISSSYGY
TYYADSVKGR FTISADTSKN TAYLQMNSLR AEDTAVYYCA RTVRGSKKPY FSGWAMDYWG
QGTLVTVSS
CDCP1-001 V_{L} (SEQ ID NO: 78)
DIQMTQSPS SLSASVGDRV TITCRASQSV SSAVAWYQQK PGKAPKLLIY SASSLYSGVP
SRFSGSRSGT DFTLTISSLQ PEDFATYYCQ QSSYSLITFG QGTKVEIK
^{∧}CDR sequences are underlined

Similarly, the combination of any 4 VH FR regions with VH CDRs1-3 from Table A-1, in proper order, would make up a VH chain for an anti-CDCP1 antibody or antibody fragment that is within the scope of the present disclosure. Furthermore, the combination of any 4 VL FRs1, VL CDR1 (SEQ ID NO:11), CDR2 (SEQ ID NO:13) and VL CDR3 from Table A-1, in proper order, make up the VL chain for an anti-CDCP1 antibody or antibody fragment that is within the scope of the present disclosure. Accordingly, the disclosure provides humanized antibodies or antibody fragments. For example, humanized antibodies or antibody fragments may be generated by inserting CDRs generated in animals into framework regions from other human antibodies or antibody fragments. Thus, the framework regions of the antibody or antibody fragments need not be the identical amino acid sequences of the framework regions of the CDCP1-001 antibody.

In certain embodiments, an antibody or antibody fragment provided herein comprises:
the VH amino acid sequence shown in SEQ ID NO:57, and the VL amino acid sequence shown in SEQ ID NO:58;
the VH amino acid sequence shown in SEQ ID NO:59, and the VL amino acid sequence shown in SEQ ID NO:60;
the VH amino acid sequence shown in SEQ ID NO:61, and the VL amino acid sequence shown in SEQ ID NO:62;
the VH amino acid sequence shown in SEQ ID NO:63, and the VL amino acid sequence shown in SEQ ID NO:64;
the VH amino acid sequence shown in SEQ ID NO:65, and the VL amino acid sequence shown in SEQ ID NO:66;
the VH amino acid sequence shown in SEQ ID NO:67, and the VL amino acid sequence shown in SEQ ID NO:68;
the VH amino acid sequence shown in SEQ ID NO:69, and the VL amino acid sequence shown in SEQ ID NO:70;
the VH amino acid sequence shown in SEQ ID NO:71, and the VL amino acid sequence shown in SEQ ID NO:72;
the VH amino acid sequence shown in SEQ ID NO:73, and the VL amino acid sequence shown in SEQ ID NO:74;
the VH amino acid sequence shown in SEQ ID NO:75, and the VL amino acid sequence shown in SEQ ID NO:76; or
the VH amino acid sequence shown in SEQ ID NO:77, and the VL amino acid sequence shown in SEQ ID NO:78.

In certain embodiments, an antibody or antibody fragment provided herein comprises the VH CDR1, CDR2, and CDR3 amino acid sequences shown in SEQ ID NO:1 above, combined with the VL CDR1, CDR2 and CDR3 sequences shown in SEQ ID NO:9 above. Optionally, the VL CDR3 shown in SEQ ID NO:9 may be substituted with one of the VL CDR3 sequences shown in Table A-1. Thus, in specific embodiments, provided herein are antibodies or antibody fragments that comprise:
VH CDR1, CDR2 and CDR3 comprising the amino acid sequence of SEQ ID NO:3, SEQ ID NO:5 and SEQ ID NO:7, and VL CDR1, CDR2 and CDR3 comprising the amino acid sequence of SEQ ID NO:11, SEQ ID NO:13 and SEQ ID NO:15;
VH CDR1, CDR2 and CDR3 comprising the amino acid sequence of SEQ ID NO:17, SEQ ID NO:18 and SEQ ID NO:19, and VL CDR1, CDR2 and CDR3 comprising the amino acid sequence of SEQ ID NO:11, SEQ ID NO:13 and SEQ ID NO:20;
VH CDR1, CDR2 and CDR3 comprising the amino acid sequence of SEQ ID NO:21, SEQ ID NO:22 and SEQ ID NO:23, and VL CDR1, CDR2 and CDR3 comprising the amino acid sequence of SEQ ID NO: 11, SEQ ID NO:13 and SEQ ID NO:24;
VH CDR1, CDR2 and CDR3 comprising the amino acid sequence of SEQ ID NO:25, SEQ ID NO:26 and SEQ ID NO:27, and VL CDR1, CDR2 and CDR3 comprising the amino acid sequence of SEQ ID NO:11, SEQ ID NO:13 and SEQ ID NO:28;
VH CDR1, CDR2 and CDR3 comprising the amino acid sequence of SEQ ID NO 29, SEQ ID NO:30 and SEQ ID NO:31, and VL CDR1, CDR2 and CDR3 comprising the amino acid sequence of SEQ ID NO:11, SEQ ID NO:13 and SEQ ID NO:32;
VH CDR1, CDR2 and CDR3 comprising the amino acid sequence of SEQ ID NO:33, SEQ ID NO:34 and SEQ ID NO:35, and VL CDR1, CDR2 and CDR3 comprising the amino acid sequence of SEQ ID NO:11, SEQ ID NO:13 and SEQ ID NO:36;
VH CDR1, CDR2 and CDR3 comprising the amino acid sequence of SEQ ID NO:37, SEQ ID NO:38 and SEQ ID NO:39, and VL CDR1, CDR2 and CDR3 comprising the amino acid sequence of SEQ ID NO:11, SEQ ID NO:13 and SEQ ID NO:40;
VH CDR1, CDR2 and CDR3 comprising the amino acid sequence of SEQ ID NO:41, SEQ ID NO:42 and SEQ ID NO:43, and VL CDR1, CDR2 and CDR3 comprising the amino acid sequence of SEQ ID NO:11, SEQ ID NO:13 and SEQ ID NO:44;
VH CDR1, CDR2 and CDR3 comprising the amino acid sequence of SEQ ID NO:45, SEQ ID NO:46 and SEQ ID NO:47, and VL CDR1, CDR2 and CDR3 comprising the amino acid sequence of SEQ ID NO:11, SEQ ID NO:13 and SEQ ID NO:48;
VH CDR1, CDR2 and CDR3 comprising the amino acid sequence of SEQ ID NO:49, SEQ ID NO:50 and SEQ ID NO:51, and VL CDR1, CDR2 and CDR3 comprising the amino acid sequence of SEQ ID NO: 11, SEQ ID NO:13 and SEQ ID NO:52; or
VH CDR1, CDR2 and CDR3 comprising the amino acid sequence of SEQ ID NO:53, SEQ ID NO:54 and SEQ ID NO:55, and VL CDR1, CDR2 and CDR3 comprising the amino acid sequence of SEQ ID NO: 11, SEQ ID NO:13 and SEQ ID NO:56.

In some embodiments, an antibody or antibody fragment provided herein comprises:
the VH amino acid sequence selected from the group consisting of SEQ ID NO:57, SEQ ID NO: 59, SEQ ID NO:61, SEQ ID NO:63, SEQ ID NO:65, SEQ ID NO:67, SEQ ID NO:69, SEQ ID NO: 71, SEQ ID NO:73, SEQ ID NO:75, and SEQ ID NO:77; and
the VL amino acid sequence selected from the group consisting of SEQ ID NO:58, SEQ ID NO: 60, SEQ ID NO:62, SEQ ID NO:64, SEQ ID NO:66, SEQ ID NO:68, SEQ ID NO: 70, SEQ ID NO:72, SEQ ID NO:74, SEQ ID NO:76, and SEQ ID NO:78. For instance, an antibody or antibody fragment provided herein may comprise the VH amino acid sequence of SEQ ID NO:57, and the VL amino acid sequence selected from the group consisting of SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, and SEQ ID NO:78. Similarly, an antibody or antibody fragment provided herein may comprise the VL amino acid sequence of SEQ ID NO:58, and the VH amino acid sequence selected from the group consisting of SEQ ID NO:1, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, and SEQ ID NO:77.

In other embodiments, any of the antibodies provided herein may be a part of a bispecific or multispecific antibody. The bispecific or multispecific antibody may have both, or all, binding domains specific for CDCP1 (e.g., a CUB domain of CDCP1). For instance, a bispecific antibody may comprise VH and VL from any combination of two of the anti-CDCP1-specific Fab sequences shown in Table A-2. Alternatively, the bispecific or multispecific antibody may have a single binding domain specific for CDCP1 (e.g., a CUB domain of CDCP1), and one or more domains specific for a second antigen. In similar fashion, a bispecific antibody can comprise VH and VL from any other combination two of the anti-CDCP1-specific Fab sequences provided herein. The CDCP1-binding domains of the present disclosure may be contained in other antibody formats, including but not limited to monospecific Fab2, bispecific Fab2, trispecific Fab3, monovalent IgGT, bispecific diabody, trispecific triabody, scFv-Fc, minibody, etc.

In still further embodiments, the CDCP1-binding domains of the present disclosure may form part of a “T-cell engager” comprising a CD3 binding domain for a linking a T cell to a CDCP1+ cancer cell (anti-CDCP1xanti-CD3). Alternatively, the CDCP1-binding domains of the present disclosure may form part of a “NK-cell engager” comprising a NKG2D or CD16 binding domain for linking a NK cell to a CDCP1+ cancer cell (anti-CDCP1xanti-NKG2D or anti-CD16). Additionally, the CDCP1-binding domains of the present disclosure may form part of an ectodomain of a chimeric T cell receptor.

As used herein with respect to polypeptides, the term “substantially pure” means that the polypeptides are essentially free of other substances with which they may be found in nature or in vivo systems to an extent practical and appropriate for their intended use. In particular, the polypeptides are sufficiently pure and are sufficiently free from other biological constituents of their host cells so as to be useful in, for example, generating antibodies, sequencing, or producing pharmaceutical preparations. By techniques well known in the art, substantially pure polypeptides may be produced in light of the polynucleotide and amino acid sequences disclosed herein. Because a substantially purified polypeptide of the disclosure may be admixed with a pharmaceutically acceptable carrier in a pharmaceutical preparation, the polypeptide may comprise only a certain percentage by weight of the preparation. The polypeptide is nonetheless substantially pure in that it has been substantially separated from the substances with which it may be associated in living systems.

As used herein, “sequence identity” is a measure of the identity of nucleotide sequences or amino acid sequences compared to a reference nucleotide or amino acid sequence. A polypeptide having an amino acid sequence at least, for example, about 95% “sequence identity” to a reference an amino acid sequence, e.g., SEQ ID NO: 1, is understood to mean that the amino acid sequence of the polypeptide is identical to the reference sequence except that the amino acid sequence may include up to about five modifications per each 100 amino acids of the reference amino acid sequence. In other words, to obtain a peptide having at least about 95% sequence identity to a reference amino acid sequence, up to about 5% of the amino acid residues of the reference sequence may be deleted or substituted with another amino acid or a number of amino acids up to about 5% of the total amino acids in the reference sequence may be inserted into the reference sequence. These modifications of the reference sequence may occur at the N-terminus or C-terminus positions of the reference amino acid sequence or anywhere between those terminal positions, interspersed either individually among amino acids in the reference sequence or in one or more contiguous groups within the reference sequence.

In general, the sequences are aligned so that the highest order match is obtained. “Sequence identity” per se has an art-recognized meaning and can be calculated using well known techniques. In one embodiment of the present disclosure, the algorithm used to determine sequence identity between two or more polypeptides is BLASTP. In another embodiment of the present disclosure, the algorithm used to determine sequence identity between two or more polypeptides is FASTDB (Brutlag, Comp. App. Biosci. 6:237-245, 1990). In a FASTDB sequence alignment, the query and reference sequences are amino sequences. The result of sequence alignment is in percent sequence identity. In one embodiment, parameters that may be used in a FASTDB alignment of amino acid sequences to calculate percent sequence identity include, but are not limited to: Matrix=PAM, k-tuple=2, Mismatch Penalty=1, Joining Penalty=20, Randomization Group Length=0, Cutoff Score=1, Gap Penalty=5, Gap Size Penalty 0.05, Window Size=500 or the length of the subject amino sequence, whichever is shorter.

If the reference sequence is shorter or longer than the query sequence because of N-terminus or C-terminus additions or deletions, but not because of internal additions or deletions, a manual correction can be made, because the FASTDB program does not account for N-terminus and C-terminus truncations or additions of the reference sequence when calculating percent sequence identity. For query sequences truncated at the N- or C-termini, relative to the reference sequence, the percent sequence identity is corrected by calculating the number of residues of the query sequence that are N-and C-terminal to the reference sequence that are not matched/aligned, as a percent of the total bases of the query sequence. The results of the FASTDB sequence alignment determine matching/alignment. The alignment percentage is then subtracted from the percent sequence identity, calculated by the above FASTDB program using the specified parameters, to arrive at a final percent sequence identity score. This corrected score can be used for the purposes of determining how alignments “correspond” to each other, as well as percentage sequence identity. Residues of the reference sequence that extend past the N-or C-termini of the query sequence may be considered for the purposes of manually adjusting the percent sequence identity score. That is, residues that are not matched/aligned with the N-or C-termini of the comparison sequence may be counted when manually adjusting the percent sequence identity score or alignment numbering.

For example, a 90 amino acid residue query sequence is aligned with a 100 residue reference sequence to determine percent identity. The deletion occurs at the N-terminus of the query sequence and therefore, the FASTDB alignment does not show a match/alignment of the first 10 residues at the N-terminus. The 10 unpaired residues represent 10% of the reference sequence (number of residues at the N- and C-termini not matched/total number of residues in the reference sequence) so 10% is subtracted from the percent sequence identity score calculated by the FASTDB program. If the remaining 90 residues were perfectly matched (100% alignment) the final percent sequence identity would be 90% (100% alignment−10% unmatched overhang). In another example, a 90 residue query sequence is compared with a 100 reference sequence, except that the deletions are internal deletions. In this case the percent sequence identity calculated by FASTDB is not manually corrected, since there are no residues at the N- or C-termini of the subject sequence that are not matched/aligned with the query. In still another example, a 110 amino acid query sequence is aligned with a 100 residue reference sequence to determine percent sequence identity. The addition in the query occurs at the N-terminus of the query sequence and therefore, the FASTDB alignment may not show a match/alignment of the first 10 residues at the N-terminus. If the remaining 100 amino acid residues of the query sequence have 95% sequence identity to the entire length of the reference sequence, the N-terminal addition of the query would be ignored and the percent identity of the query to the reference sequence would be 95%.

As used here, the term “conservative substitution” denotes the replacement of an amino acid residue by another biologically similar residue. Conservative substitution for this purpose may be defined as set out in the tables below. Amino acids can be classified according to physical properties and contribution to secondary and tertiary protein structure as shown in Table B and Table C. A conservative substitution is recognized in the art as a substitution of one amino acid for another amino acid that has similar properties, such as a substitution of one amino acid for another amino acid in the same row of Table B or Table C. Exemplary conservative substitutions are set out below in Table D.

TABLE B
Amino Acid Classification - 5 Groups

| Side Chain Characteristic | Amino Acid |
|---|---|
| Aliphatic | |
| Non-polar | Gly, Ala, Pro, Iso, Leu, Val |
| Polar-uncharged | Cys, Ser, Thr, Met, Asn, Gln |
| Polar-charged | Asp, Glu, Lys, Arg |
| Aromatic | His, Phe, Trp, Tyr |
| Other | Asn, Gln, Asp, Glu |

TABLE C
Amino Acid Classification - 10 Groups

| Side Chain Characteristic | Amino Acid |
|---|---|
| Non-polar (hydrophobic) | |
| Aliphatic: | Ala, Leu, Iso, Val, Pro |
| Aromatic: | Phe, Trp |
| Sulfur-containing: | Met |
| Borderline: | Gly |
| Uncharged-polar | |
| Hydroxyl: | Ser, Thr, Tyr |
| Amides: | Asn, Gln |
| Sulfhydryl: | Cys |
| Borderline: | Gly |
| Positively Charged (Basic): | Lys, Arg, His |
| Negatively Charged (Acidic) | Asp, Glu |

TABLE D
Conservative Amino Acid Substitutions

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

In select embodiments, the disclosure is directed to antibodies or antibody fragments where the amino acid sequence of one or more framework regions is mutated. In one specific embodiment, the mutations in the one or more framework regions is a conserved substitution. In more specific embodiments, the antibodies or antibody fragments of the present disclosure comprise an amino acid sequence having at least 80%, 85%, 90%, 95%, 96%, 97%, 98%, 99% or 100% sequence identity to any one of the amino acid sequences of SEQ ID NO:2, SEQ ID NO:4, SEQ ID NO:6, SEQ ID NO:8, SEQ ID NO: 10, SEQ ID NO:12, SEQ ID NO:14, and SEQ ID NO:16.

Likewise, in some embodiments, the disclosure is directed to antibodies or antibody fragments that have a high level of identity to the VH and VL amino acid sequences of any one of exemplary antibodies CDCP1-001 to CDCP1-011. In specific embodiments, the antibodies or antibody fragments of the present disclosure comprise an amino acid sequence having at least 90%, 95%, 96%, 97%, 98%, or 99% sequence identity to any one of the amino acid sequences of SEQ ID NOs:57-78.

### 2. Radiolabeled Antibodies

The disclosure also provides for antibodies or antibody fragments that are labeled with a radionuclide (also referred to herein as a radioisotope). In some embodiments, the disclosure provides antibody-radionuclide conjugates (ARCs). As used herein, an ARC need not be composed of an entire, intact antibody and instead can be composed of any one of the antibody fragments disclosed herein. In some embodiments, the radioisotope-labeled antibody or antibody fragment has the formula: Radioisotope-Linker-Antibody (R *-L-Ab), wherein R* is a radioisotope, L is a linker, and Ab is an antibody or antibody fragment that specifically binds CUB domain-containing protein 1 (CDCP1) ectodomain. In some embodiments, the linker is a macrocyclic chelator. In an exemplary embodiment, the macrocyclic chelator comprises tetraxetan. In some embodiments, the radioisotope is a therapeutic radioisotope. In other embodiments, the radioisotope is a diagnostic radioisotope.

As used herein, the term “therapeutic radioisotope” refers to an alpha-emitter and/or a beta-minus-emitter, preferably with a half-life of hours to days. In some embodiments, the therapeutic radioisotope is selected from the group consisting of ⁶⁷Cu, ⁹⁰Y, ¹³¹I, ¹⁵³Sm, ¹⁷⁷Lu, ²¹¹At, ²¹²Ph, ²²³Ra, ²²⁵Ac, and ²²⁷Th. In an exemplary embodiment, the therapeutic radioisotope is ¹⁷⁷Lu.

The term “diagnostic radioisotope” as used herein, refers to a positron-emitter, preferably with a half-life of minutes to hours. In some embodiments, the diagnostic radioisotope is selected from the group consisting of ¹⁸F, ⁴⁴Sc ⁶⁴Cu, ⁶⁸Ga, ⁸⁹Zr, and ¹²⁴I. In an exemplary embodiment, the diagnostic radioisotope is ⁸⁹Zr.

In some preferred embodiments, the antibody or antibody fragment comprises the amino acid sequences of the light chain CDRs of 4A06 are set forth as SEQ ID NO:11, SEQ ID NO:13 and SEQ ID NO:20, and the amino acid sequences of the heavy chain CDRs of 4A06 are set forth as SEQ ID NO:17, SEQ ID NO:18, and SEQ ID NO:19. In some preferred embodiments, the antibody or antibody fragment comprises the amino acid sequence of the heavy chain variable domain (VH) of 4A06 are set forth as SEQ ID NO:57, and the amino acid sequence of the light chain variable domain (VL) of 4A06 is set forth as SEQ ID NO:58. The recombinant monoclonal antibody 4A06 is also referred to herein as CDCP1-002. In some embodiments, the antibody is an IgG₁ antibody.

### 3. Pharmaceutical Compositions

Further provided are pharmaceutical compositions comprising the radiolabeled antibodies or antibody fragments described. The pharmaceutical compositions comprise at least one radiolabeled antibody or antibody fragment of the present disclosure and a pharmaceutical excipient. The pharmaceutical compositions may be administered, or may be formulated to be administered, by parenteral administration, for example, intravenous administration. Intravenous administration may be done by injection or infusion.

The present compositions will contain a therapeutically effective amount of the radiolabeled antibodies or antibody fragments, together with a suitable amount of a pharmaceutically acceptable vehicle so as to provide the form for proper administration to a patient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the disclosure is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds of the disclosure and pharmaceutically acceptable vehicles should be sterile. Water is one example of a vehicle when the compound of the disclosure is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The pharmaceutical compositions may further contain one or more auxiliary substance, such as wetting or emulsifying agents, pH buffering agents, or adjuvants to enhance the effectiveness thereof.

In another embodiment, the compounds and/or compositions of the disclosure (radiolabeled antibodies) are formulated in accordance with routine procedures as a pharmaceutical composition adapted for intravenous administration to mammals, including humans. Typically, compounds and/or compositions of the disclosure for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the disclosure is to be administered by infusion, it can be dispensed, for example, with an infusion bottle containing sterile pharmaceutical grade water or saline. Where the compound of the disclosure is administered by injection, an ampoule of sterile water for injection or saline can be provided so that the ingredients may be mixed prior to administration.

The amount of a compound of the disclosure that will be effective in the treatment of a particular disorder or condition disclosed herein will depend on the nature of the disorder or condition and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. In specific embodiments of the disclosure, the oral dose of at least one compound of the present disclosure is about 0.01 milligram to about 100 milligrams per kilogram body weight, or from about 0.1 milligram to about 50 milligrams per kilogram body weight, or from about 0.5 milligram to about 20 milligrams per kilogram body weight, or from about 1 milligram to about 10 milligrams per kilogram body weight.

Suitable dosage ranges for parenteral, for example, intravenous (IV) administration are 0.01 milligram to 100 milligrams radiolabeled antibody per kilogram body weight, 0.1 milligram to 35 milligrams per kilogram body weight, and 1 milligram to 10 milligrams per kilogram body weight. In other embodiments, a composition of the disclosure for parenteral, for example, intravenous administration includes about 0.001 milligram to about 2000 milligrams of a compound of the disclosure, from about 0.01 milligram to about 1000 milligrams of a compound of the disclosure, from about 0.1 milligram to about 500 milligrams of a compound of the disclosure, or from about 1 milligram to about 200 milligrams of a compound of the disclosure.

The disclosure also provides pharmaceutical packs or kits comprising one or more containers filled with one or more of the radiolabeled antibodies or antibody fragments of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration.

A therapeutically effective amount of the antibodies or antibody fragments in the composition should be administered, in which “a therapeutically effective amount” is defined as an amount that is sufficient to produce a desired prophylactic, therapeutic or ameliorative response in a subject. The amount needed will vary depending upon the antibodies or antibody fragments used and the species and weight of the subject to be administered but may be ascertained using standard techniques.

### 4. Methods of Treatment

The present disclosure provides methods for treating CUB domain-containing protein 1 (CDCP1)-positive bladder cancer, comprising: administering to a subject in need thereof a therapeutic radioisotope-labeled antibody or antibody fragment that specifically binds CDCP1 ectodomain to treat the bladder cancer, wherein the therapeutic radioisotope is an alpha-emitter and/or a beta-minus-emitter. Additionally, the present disclosure provides kits for a treating CUB domain-containing protein 1 (CDCP1)-positive bladder cancer, comprising: a therapeutic radioisotope-labeled antibody or antibody fragment that specifically binds CDCP1 ectodomain; and instructions for use of the therapeutic radioisotope-labeled antibody or antibody fragment to treat the bladder cancer. In some embodiments, the therapeutic radioisotope is selected from the group consisting of ⁶⁷Cu, ⁹⁰Y, ¹³¹I, ¹⁵³Sm, ¹⁷⁷Lu, ²¹¹At, ²¹²Pb, ²²³Ra, ²²⁵Ac, and ²²⁷Th. In exemplary embodiments, the therapeutic radioisotope is ¹⁷⁷Lu.

When expressed as units of weight, an effective amount of a therapeutic radioisotope-labeled antibody or antibody fragment for treating CDCP1-positive bladder cancer is in the range of about 0.1 μg to about 100 mg, preferably from about 1 μg to about 10 mg, preferably from about 10 μg to about 1 mg, or preferably from about 50 μg to about 500 μg. When expressed as units of radioactivity, an effective amount of a therapeutic radioisotope-labeled antibody or antibody fragment for treating CDCP1-positive bladder cancer is in the range of from about 1 μCi to about 10 mCi, or preferably from about 10 μCi to about 1 mCi, or preferably from about 50 μCi to about 500 μCi.

In some embodiments, the bladder cancer is metastatic bladder cancer (mBC). In some embodiments, the bladder cancer is muscle-invasive bladder cancer (MIBC). In some embodiments, the bladder cancer is a basal/squamous (Ba/Sq) subtype of MIBC. In some embodiments, cells of the bladder cancer are TROP2-negative and/or NECTIN4 negative. In some embodiments, cells of the bladder cancer express little to no (negligible levels) of TROP2 and/or NECTIN4.

Further provided are methods of suppressing or inhibiting the growth of, or reducing the growth rate of, bladder cancer cells that express CDCP1, comprising contacting the cells with, or administering to the cells, a pharmaceutically effective amount of a composition comprising at least one of the radiolabeled anti-CDCP1 antibodies or antibody fragments of the present disclosure. In certain specific embodiments, the bladder cancer cells harbor a KRAS mutation.

Also provided herein is a method of treating a subject having a bladder cancer, e.g., a bladder cancer expressing CDCP1 protein, comprising administering to the subject a therapeutically effective amount of a radiolabeled anti-CDCP1 antibody or antibody fragment provided herein. Further provided herein is a method of reducing the likelihood of bladder cancer metastasis, comprising administering to a subject in need of such treatment a therapeutically effective amount of a composition comprising a radiolabeled anti-CDCP1 antibody or antibody fragment provided herein. In certain specific embodiments, the bladder cancer comprises a KRAS mutation.

The subject is preferably a human but can be another mammal. The methods of treatment or prophylaxis of bladder cancer comprise administering to a subject in need of treatment thereof a pharmaceutically effective amount of a composition comprising at least one of the radiolabeled anti-CDCP1 antibodies or antibody fragments provided herein.

As used herein and unless otherwise indicated, the terms “cancer” or “cancer cell” refer to abnormal cell growth or proliferation that may or may not include spontaneous or induced phenotypic changes. As used herein, “cancer” includes but is not limited to such abnormal conditions as hypertrophy, neoplasia, hyperplasia, benign and malignant cancer. As used herein, the term “tumor” is a general term that includes hypertrophies, neoplasias, hyperplasias, benign cancers and malignant cancers. Accordingly, certain embodiments of the present disclosure include but are not limited to treating a hypertrophy, a neoplasia, a hyperplasia, a benign or a malignant cancer in a subject. In additional embodiments, the present disclosure is directed to preventing or reducing the likelihood of metastasis and/or recurrence of a hypertrophy, a neoplasia, a hyperplasia, a benign or a malignant cancer within a subject comprising administering at least one compound of the present disclosure to the subject. For example, at least one compound of the present disclosure may be administered after tumor resection/removal/ablation, etc. to reduce the likelihood of recurrence of the tumor in the subject. In another example, at least one compound of the present disclosure may be administered to reduce the likelihood of metastasis of the tumor in the subject.

As used herein, “treatment” or “treating” refers to an amelioration of a disease or disorder, or at least one detectable symptom thereof, or amelioration of at least one measurable physical parameter, not necessarily discernible by the patient. “Treatment” or “treating” may also refer to inhibiting the progression of a disease or disorder, either physically, e.g., stabilization of a discernible symptom, physiologically, e.g., stabilization of a physical parameter, or both, or to delaying the onset of a disease or disorder.

As used herein, the term “prevent,” as it relates to tumors and/or abnormal cell growth, indicates that the antibodies or antibody fragments of the present disclosure is administered to a subject to at least partially inhibit or reduce the likelihood of growth, division, spread, or proliferation of cancer cells. Of course, the term “prevent” also encompasses prohibiting entirely the emergence of new tumors or any of the associated symptoms from detectably appearing. Thus, a subject may be “pretreated,” by administering the one or more the antibodies or antibody fragments of the present disclosure to prevent tumors from arising. The phrase “preventing the progression,” as it relates to tumors, is used to mean a procedure designed to at least partially inhibit the detectable appearance of one or more additional tumors or aberrant cell growth in a patient already exhibiting one or more symptoms of the presence of a tumor or aberrant cell growth, and is also used to mean at least partially prohibiting the already-present symptoms of cancer from worsening in the subject.

As used herein, the term “administer” and “administering” are used to mean introducing at least one of the antibodies or antibody fragments to the cells in close enough proximity that the antibodies or antibody fragments can exert an effect on the cells. For example, “administer,” in an in vivo setting refers to introducing the antibodies or antibody fragments to the subject in need of treatment or prophylactic treatment thereof. In an in vitro, e.g., cell culture, setting, the term “administer” can mean to introduce the antibodies or antibody fragments into the cell culture environment such that the antibodies or antibody fragments contact the cells. When administration is for the purpose of treatment of a subject in need of treatment thereof, the antibodies or antibody fragments can be provided at, or after the diagnosis of an abnormal cell growth, such as a tumor. The therapeutic administration of the antibodies or antibody fragments serves to inhibit cell growth of the tumor or abnormal cell growth.

### 5. Methods of Detection

The disclosure also provides methods for assessing CUB domain-containing protein 1 (CDCP1) expression in vivo, comprising: (a) administering a diagnostic radioisotope-labeled antibody or antibody fragment that specifically binds the CDCP1 ectodomain to a subject having bladder cancer; and (b) visualizing binding of the antibody or antibody fragment to cells of the subject by radionuclide imaging to assess CDC1 expression, wherein the diagnostic radioisotope is a positron-emitter. Additionally, the present disclosure provides kits for assessing CUB domain-containing protein 1 (CDCP1) expression in vivo, comprising: a diagnostic radioisotope-labeled antibody or antibody fragment that specifically binds CDCP1 ectodomain; and instructions for use of the diagnostic radioisotope-labeled antibody or antibody fragment to visualize binding of the diagnostic radioisotope-labeled antibody or antibody fragment to cells of the subject by radionuclide imaging. The positron-emitter may be a beta-plus-emitter and/or a gamma-emitter. In some embodiments, the diagnostic radioisotope is selected from the group consisting of ¹⁸F, ⁴⁴Sc ⁶⁴Cu, ⁶⁸Ga, ⁸⁹Zr, and ¹²⁴I. In exemplary embodiments, the diagnostic radioisotope is ⁸⁹Zr. In some embodiments, the radionuclide imagining comprises positron emission tomography (PET). In some embodiments, the radionuclide imaging comprises single-photon emission computed tomography (SPECT).

When expressed as units of weight, an effective amount of a diagnostic radioisotope-labeled antibody or antibody fragment for assessing CDCP1 expression in vivo is in the range of about 1 μg to about 10 mg, preferably from about 10 μg to about 1 mg, or preferably from about 50 μg to about 500 μg. When expressed as units of radioactivity, an effective amount of a diagnostic radioisotope-labeled antibody or antibody fragment is in the range of from about 1 μCi to about 10 mCi, or preferably from about 10 μCi to about 1 mCi, or preferably from about 50 μCi to about 500 μCi.

In some embodiments, the bladder cancer is metastatic bladder cancer (mBC). In some embodiments, the bladder cancer is muscle-invasive bladder cancer (MIBC). In some embodiments, the bladder cancer is a basal/squamous (Ba/Sq) subtype of MIBC. In some embodiments, cells of the bladder cancer are TROP2-negative and/or NECTIN4 negative. In some embodiments, cells of the bladder cancer express little to no (negligible levels) of TROP2 and/or NECTIN4.

### ENUMERATED EMBODIMENTS

1. A method for treating CUB domain-containing protein 1 (CDCP1)-positive bladder cancer, comprising:
administering to a subject in need thereof a therapeutic radioisotope-labeled antibody or antibody fragment that specifically binds CDCP1 ectodomain to treat the bladder cancer, wherein the therapeutic radioisotope is an alpha-emitter and/or a beta-minus-emitter.

2. The method of embodiment 1, wherein the antibody or antibody fragment comprises a light chain CDR1 comprising SEQ ID NO:11, a light chain CDR2 comprising SEQ ID NO:13, and:
(a) a light chain CDR3 comprising SEQ ID NO:20, a heavy chain CDR1 comprising SEQ ID NO:17, a heavy chain CDR2 comprising SEQ ID NO:18, and a heavy chain CDR3 comprising SEQ ID NO:19;
(b) a light chain CDR3 comprising SEQ ID NO:24, a heavy chain CDR1 comprising SEQ ID NO:21, a heavy chain CDR2 comprising SEQ ID NO:22, and a heavy chain CDR3 comprising SEQ ID NO:23;
(c) a light chain CDR3 comprising SEQ ID NO:28, a heavy chain CDR1 comprising SEQ ID NO:25, a heavy chain CDR2 comprising SEQ ID NO:26, and a heavy chain CDR3 comprising SEQ ID NO:27;
(d) a light chain CDR3 comprising SEQ ID NO:32, a heavy chain CDR1 comprising SEQ ID NO:29, a heavy chain CDR2 comprising SEQ ID NO:30, and a heavy chain CDR3 comprising SEQ ID NO:31;
(e) a light chain CDR3 comprising SEQ ID NO:36, a heavy chain CDR1 comprising SEQ ID NO:33, a heavy chain CDR2 comprising SEQ ID NO:34, and a heavy chain CDR3 comprising SEQ ID NO:35;
(f) a light chain CDR3 comprising SEQ ID NO:40, a heavy chain CDR1 comprising SEQ ID NO:37, a heavy chain CDR2 comprising SEQ ID NO:38, and a heavy chain CDR3 comprising SEQ ID NO:39;
(g) a light chain CDR3 comprising SEQ ID NO:44, a heavy chain CDR1 comprising SEQ ID NO:41, a heavy chain CDR2 comprising SEQ ID NO:42, and a heavy chain CDR3 comprising SEQ ID NO:43;
(h) a light chain CDR3 comprising SEQ ID NO:48, a heavy chain CDR1 comprising SEQ ID NO:45, a heavy chain CDR2 comprising SEQ ID NO:46, and a heavy chain CDR3 comprising SEQ ID NO:47;
(i) a light chain CDR3 comprising SEQ ID NO:52, a heavy chain CDR1 comprising SEQ ID NO:49, a heavy chain CDR2 comprising SEQ ID NO:50, and a heavy chain CDR3 comprising SEQ ID NO:51; or
(j) a light chain CDR3 comprising SEQ ID NO:56, a heavy chain CDR1 comprising SEQ ID NO:53, a heavy chain CDR2 comprising SEQ ID NO:54, and a heavy chain CDR3 comprising SEQ ID NO:55.

3. The method of embodiment 1, wherein the antibody or antibody fragment comprises light chain CDRs of SEQ ID NO:11, SEQ ID NO:13 and SEQ ID NO:20, and heavy chain CDRs of SEQ ID NO:17, SEQ ID NO:18, and SEQ ID NO:19.

4. The method of embodiment 1, wherein the antibody or antibody fragment comprises a heavy chain variable domain (VH) and a light chain variable domain (VL), and the VH comprises an amino acid sequence having at least 95% sequence identity to SEQ ID NO:57, and the VL comprises an amino acid sequence having at least 95% sequence identity to SEQ ID NO:58.

5. The method of embodiment 1, wherein the antibody or antibody fragment comprises a heavy chain variable domain (VH) and a light chain variable domain (VL), and the VH comprises the amino acid sequence of SEQ ID NO:57, and the VL comprises the amino acid sequence of SEQ ID NO:58.

6. The method of any one of embodiments 1-5, wherein the antibody or antibody fragment is a recombinant IgG₁ antibody.

7. The method of any one of embodiments 1-6, wherein the therapeutic radioisotope is selected from the group consisting of ⁶⁷Cu, ⁹⁰Y, ¹³¹I, ¹⁵³Sm, ¹⁷⁷Lu, ²¹¹At, ²¹²Pb, ²²³Ra, ²²⁵Ac, and ²²⁷Th.

8. The method of any one of embodiments 1-6, wherein the therapeutic radioisotope is ¹⁷⁷Lu.

9. The method of any one of embodiments 1-8, wherein the bladder cancer is muscle-invasive bladder cancer (MIBC)

10. The method of embodiment 9, wherein the bladder cancer is a basal/squamous (Ba/Sq) subtype of MIBC.

11. The method of any one of embodiments 1-10, wherein cells of the bladder cancer are TROP2-negative and/or NECTIN4-negative, optionally wherein cells of the bladder cancer are NECTIN4-negative.

12. The method of any one of embodiments 1-11, further comprising prior to administering the therapeutic radioisotope-labeled antibody or antibody fragment: determining the bladder cancer is CDCP1-positive by immunohistochemistry analysis of a bladder cancer biopsy obtained from the subject.

13. The method of any one of embodiments 1-12, further comprising prior to administering the therapeutic radioisotope-labeled antibody or antibody fragment:
determining the bladder cancer is CDCP1-positive by radionuclide imaging following administering a diagnostic radioisotope-labeled antibody or antibody fragment that specifically binds the CDCP1 ectodomain to the subject, wherein the diagnostic radioisotope is a positron-emitter.

14. The method of embodiment 13, wherein the diagnostic radioisotope is selected from the group consisting of ₁₈F, ₄₄Sc ₆₄Cu, ₆₈Ga, ₈₉Zr, and ₁₂₄I.

15. The method of embodiment 13, wherein the diagnostic radioisotope is ₈₉Zr.

16. The method of any one of embodiments 13-15, wherein the radionuclide imagining comprises positron emission tomography (PET) or single-photon emission computed tomography (SPECT).

17. The method of any one of embodiments 1-16, wherein one or both of the therapeutic radioisotope-labeled antibody or antibody fragment and the diagnostic radioisotope-labeled antibody or fragment are administered intravenously.

18. The method of any one of embodiments 1-16, wherein one or both of the therapeutic radioisotope-labeled antibody or antibody fragment and the diagnostic radioisotope-labeled antibody or fragment are administered by intravesicular deliver to the bladder.

19. A method for assessing CUB domain-containing protein 1 (CDCP1) expression, comprising:
(a) administering a diagnostic radioisotope-labeled antibody or antibody fragment that specifically binds the CDCP1 ectodomain to a subject having bladder cancer; and
(b) visualizing binding of the antibody or antibody fragment to cells of the subject by radionuclide imaging to assess CDC1 expression, wherein
the diagnostic radioisotope is a positron-emitter.

20. The method of embodiment 19, wherein the antibody or antibody fragment comprises a light chain CDR1 comprising SEQ ID NO:11, a light chain CDR2 comprising SEQ ID NO:13, and:
(a) a light chain CDR3 comprising SEQ ID NO:20, a heavy chain CDR1 comprising SEQ ID NO:17, a heavy chain CDR2 comprising SEQ ID NO:18, and a heavy chain CDR3 comprising SEQ ID NO:19;
(b) a light chain CDR3 comprising SEQ ID NO:24, a heavy chain CDR1 comprising SEQ ID NO:21, a heavy chain CDR2 comprising SEQ ID NO:22, and a heavy chain CDR3 comprising SEQ ID NO:23;
(c) a light chain CDR3 comprising SEQ ID NO:28, a heavy chain CDR1 comprising SEQ ID NO: 25, a heavy chain CDR2 comprising SEQ ID NO:26, and a heavy chain CDR3 comprising SEQ ID NO:27;
(d) a light chain CDR3 comprising SEQ ID NO:32, a heavy chain CDR1 comprising SEQ ID NO:29, a heavy chain CDR2 comprising SEQ ID NO:30, and a heavy chain CDR3 comprising SEQ ID NO:31;
(e) a light chain CDR3 comprising SEQ ID NO:36, a heavy chain CDR 1 comprising SEQ ID NO:33, a heavy chain CDR2 comprising SEQ ID NO:34, and a heavy chain CDR3 comprising SEQ ID NO:35;
(f) a light chain CDR3 comprising SEQ ID NO:40, a heavy chain CDR 1 comprising SEQ ID NO:37, a heavy chain CDR2 comprising SEQ ID NO:38, and a heavy chain CDR3 comprising SEQ ID NO:39;
(g) a light chain CDR3 comprising SEQ ID NO:44, a heavy chain CDR1 comprising SEQ ID NO:41, a heavy chain CDR2 comprising SEQ ID NO:42, and a heavy chain CDR3 comprising SEQ ID NO:43;
(h) a light chain CDR3 comprising SEQ ID NO:48, a heavy chain CDR1 comprising SEQ ID NO:45, a heavy chain CDR2 comprising SEQ ID NO:46, and a heavy chain CDR3 comprising SEQ ID NO:47;
(i) a light chain CDR3 comprising SEQ ID NO:52, a heavy chain CDR1 comprising SEQ ID NO:49, a heavy chain CDR2 comprising SEQ ID NO:50, and a heavy chain CDR3 comprising SEQ ID NO:51; or
(j) a light chain CDR3 comprising SEQ ID NO:56, a heavy chain CDR1 comprising SEQ ID NO:53, a heavy chain CDR2 comprising SEQ ID NO:54, and a heavy chain CDR3 comprising SEQ ID NO:55.

21. The method of embodiment 19, wherein the antibody or antibody fragment comprises light chain CDRs of SEQ ID NO:11, SEQ ID NO:13 and SEQ ID NO:20, and heavy chain CDRs of SEQ ID NO:17, SEQ ID NO:18, and SEQ ID NO:19.

22. The method of embodiment 19, wherein the antibody or antibody fragment comprises a heavy chain variable domain (VH) and a light chain variable domain (VL), and the VH comprises an amino acid sequence having at least 95% sequence identity to SEQ ID NO:57, and the VL comprises an amino acid sequence having at least 95% sequence identity to SEQ ID NO:58.

23. The method of embodiment 19, wherein the antibody or antibody fragment comprises a heavy chain variable domain (VH) and a light chain variable domain (VL), and the VH comprises the amino acid sequence of SEQ ID NO:57, and the VL comprises the amino acid sequence of SEQ ID NO:58.

24. The method of any one of embodiments 19-23, wherein the diagnostic radioisotope is selected from the group consisting of ¹⁸F, ⁴⁴Sc ⁶⁴Cu, ⁶⁸Ga, ⁸⁹Zr, and ¹²⁴I.

25. The method of any one of embodiments 19-23, wherein the diagnostic radioisotope is ⁸⁹Zr.

26. The method of any one of embodiments 19-25, wherein the radionuclide imagining comprises positron emission tomography (PET) or single-photon emission computed tomography (SPECT).

27. The method of any one of embodiments 19-26, wherein the bladder cancer is muscle-invasive bladder cancer (MIBC)

28. The method of embodiment 27, wherein the bladder cancer is a basal/squamous (Ba/Sq) subtype of MIBC.

29. The method of any one of embodiments 19-28, wherein cells of the bladder cancer are TROP2-negative and/or NECTIN4-negative, optionally wherein cells of the bladder cancer are TROP2-negative and/or NECTIN4-negative.

30. The method of any one of embodiments 19-29, further comprising determining the subject has a CUB domain-containing protein 1 (CDCP1)-positive bladder cancer, and treating the CDCP1-positive bladder cancer according to the method of any one of embodiments 1-8.

### EXAMPLES

The examples presented herein are meant for illustrative purposes and are not intended to limit the full scope of the disclosure. The attached figures are meant to be integral parts of the specification of the disclosure.

Abbreviations: ARC (antibody radionuclide conjugate); BC (bladder cancer); CDCP1 (CUB domain-containing protein); CDR (complementarity determining region); ECD (extracellular domain); ELISA (enzyme-linked immunosorbent assay); Fab (antigen-binding antibody fragment); FACS (fluorescent activated cell sorting); FR (framework region); MFI (mean fluorescence intensity); MIBC (muscle-invasive bladder cancer); MMAF (monomethyl auristatin F); PDX (patient-derived xenograft); PET (positron emission tomography); SPECT (single-photon emission computed tomography); TRT (targeted radiotherapy); VH or V_{H} (heavy chain variable domain); VL or V_{L} (light chain variable domain); and WT (wild type).

### Example 1: Targeted Radioligand Therapy for Bladder Cancer Through Use of an Anti-CUB domain containing protein 1 (CDCP1) Antibody

This example shows that CDCP1 can be targeted on bladder cancer (BC) tumors for therapy with radiolabeled antibodies.

### Materials and Methods

Cell cultures and xenografts. HT-1376, HT-1197, 5637, TCCSUP and 639V cells were obtained from the UCSF Cell Culture Facility. UMUC-3, T-24, 253-J, UMUC-9 cells were gifts from Bradley Stohr (UCSF) and David McConkey (Pathology Core, Bladder Cancer SPORE, MD Anderson Cancer Center). Bladder cancer lines were grown in standard MEM media (Life Technologies) supplemented with 10% FBS (Seradigm). Cellular identity was authenticated by visually inspecting morphology and probing for signature expression markers on immunoblot. Mycoplasma was tested after thawing cryostocks with the MycoAlert kit (Lonza). Bladder cancer patient derived xenografts (PDX) were generated at UCSF in collaboration with the Preclinical Therapeutics Core.

Antibodies. Sotorasib and trametinib were purchased from MedChemExpress and used without further purification. The recombinant monoclonal antibody 4A06 was expressed and purified in the IgG₁ format as previously described (Martinko et al., eLIFE, 7:e31098, 2018). 4A06 (also referred to herein as CDCP1-002) binds to an extracellular epitope contained on both full length and cleaved human CDCP1. p-SCN-Bn-Deferoxamine (B-705) and p-SCN-Bn-DOTA (B-205) were purchased from Macrocyclics (Plano, TX) and directly used for conjugation to antibody. ⁸⁹Zr-oxalate was obtained from 3D Imaging, LLC (Maumelle, AR). ¹⁷⁷LuCl₃ was obtained from Oak Ridge National Laboratory. Iodine-125 was obtained from Perkin Elmer.

Patient populations and transcriptome profiling. Transcriptome data sets, namely the Sjödahl 2012 dataset, the Sjödahl 2017 dataset, TCGA dataset, and the Seiler 2017 dataset, from four retrospective publicly available cohorts of patients with muscle-invasive bladder cancer were analyzed. Briefly, the Sjödahl 2012 (n=93) dataset (obtained from NCBI Gene Expression Omnibus [GEO], accession number GSE32894) had undergone batch effect correction, quantile normalization, log2-transformation, and gene centering (Sjödahl et al., Clin Cancer Res, 18(12):3377-86, 2012). The Sjödahl 2017 (n=243) dataset (obtained from GEO, accession number GSE83586) had been pre-processed including RMA-normalization and gene centering (Sjödahl et al., J Pathol, 242 (1): 113-25, 2017). TCGA (n=406) dataset (Robertson et al., Cell, 171(3):540-556, 2017) was obtained from cBioPortal and had been normalized by RSEM and was further transformed by log2 (RSEM+1), and was renormalized to TPM when undergoing comparisons to healthy patient data (Gao et al., Sci Signal, 6(269):11, 2013) (Cerami et al., Cancer Discov, 2(5):401-4, 2012). The Seiler 2017 (n=305) dataset (obtained from GEO, accession number GSE87304) had been SCAN-normalized (Seiler et al., Eur Urol, 72(4):544-54, 2017). All cohorts underwent consensus molecular cluster subtyping as previously described (Kamoun et al., Eur Urol, 77(4):420-33, 2020). A healthy cohort from the GTEx Consortium was used to investigate normal tissue expression of CDCP1 (Consortium GT, Science, 369(6509):1318-30, 2020). All further analyses were made in the R statistical environment v4.1.1.

Histology and Immunohistochemistry. A bladder cancer tissue microarray (TMA), which contains 80 formalin-fixed, paraffin-embedded (FFPE) biopsy specimens in duplicate, was obtained from the University of British Columbia (Seiler et al., Clin Cancer Res, 25(16):5082-93, 2019). Written informed consent was obtained prior to collecting the biopsy samples. Sections of FFPE tissue were placed into the Ventana Discovery Ultra automated slide stainer. Antigen retrieval was performed using heat-inactivated antigen retrieval buffer (Tris-EDTA) according to the manufacturer instructions (Roche) and then stained with a rabbit polyclonal CDCP1 primary antibody (Cell Signaling Technology, #4115S, 1:50). Secondary antibodies (Anti-Rabbit HQ and HQ-HRP, from Ventana) were incubated for 12 min each, and DAB was used for detection for single stains. Slides were counterstained with hematoxylin per standard protocol. H-scores for CDCP1 staining were assigned by two independent pathologists (1+, 2+and 3+multiplied by the percentage), and the average of the H-scores was calculated.

Flow Cytometry. Cells were trypsinized and washed, and then incubated with either a PE-anti-CDCP1 antibody (Biolegend #324017, 1:100) or 4A06 for 30 minutes on ice. For cells incubated with unlabeled 4A06, after the 30 min incubation, the cells were washed with PBS multiple times and placed in a fluorophore conjugated secondary antibody solution for 30 minutes on ice (1:1000, 109-546-097, Jackson ImmunoResearch). Cells were collected and washed before being placed in PBS and passed through a cell strainer. Cells were analyzed using an Attune NxT Flow and data were analyzed using FlowJo software.

Immunoblot. Cell lines and PDX samples were lysed in Pierce RIPA lysis buffer (89900, ThermoFisher Scientific) with protease and phosphatase inhibitors (1861281, ThermoFisher Scientific). The samples were homogenized using a probe sonicator (Omni TH-01) and then centrifuged for 15 minutes at 15,000×g. After calculating protein concentration using a Bradford Assay kit, 15 μg of lysate was resolved via electrophoresis using a 4-12% Bis Tris gel (NW04120BOX, Invitrogen). Gels were transferred onto an Immobilon-P membrane (IPVH00010, Millipore). Membranes were blocked in 5% milk in TBST for 30 minutes at room temperature before being placed in a primary antibody solution. The primary antibodies used were anti-CDCP1 (4115, Cell Signaling, 1:1000), TROP2 (214488, abcam, 1:1000), NECTIN4 (17402S, Cell Signaling, 1:1000), and anti-beta actin (A5441, Sigma-Aldrich, 1:5000) or GAPHDH (Cell Signaling #5174). The antibodies were incubated for 1 hour at room temperature or overnight at 40 C. Membranes were then washed with TBST and incubated with a secondary antibody solution for 30 min at room temperature. Secondary antibodies used were goat anti-rabbit (65-6120, Invitrogen, 1:5000), goat anti-rat (62-6520, Invitrogen, 1:5000), or HRP-anti-rabbit (Cell Signaling Technology, #7074). Proteins were detected using West Pico Chemiluminescent Substrate for 20 seconds (34578, ThermoFisher Scientific) and then exposed to film (30-507, Blue Devil). Each immunoblot was reproduced at least once with freshly harvested protein samples.

Saturation binding assays. ¹²⁵I-4A06 was prepared as previously reported (Moroz et al., Clin Cancer Res, 6(14):3608-3615, 2020). The final yield of purified ¹²⁵I-4A06 was ˜60% (specific activity ˜1 μCi/μg) and purity was >99%. UMUC3, TCC SUP, 5637, and UMUC9 cells (0.6×10⁶ cell/well) were seeded on 12-well plates using DMEM (10% FBS). The cells were washed with PBS for the saturation binding assay. Total binding of ¹²⁵I-4A06 was determined by adding it to cell suspensions at seven concentrations from 0.025 nM to 10 nM. Non-specific binding was determined by adding 1000× cold 4A06 to ¹²⁵I-4A06/cell mixtures at three concentrations. Cells were incubated with ˜0.5 μCi ¹²⁵I-4A06 at room temperature for 1 hr, washed with PBS, and lysed by adding 1.0 M NaOH. The bound and unbound radioactive fractions were collected and measured on a Hidex Gamma counter (Turku, FI). Bmax was calculated using Prism v8.0.

Animal studies. For tumor imaging or treatment studies with BC xenografts from cell line implants, six to eight-week-old intact male athymic nu/nu mice (Charles River) were utilized. Mice were inoculated subcutaneously (˜1.5×10⁶ cells) in the flank with a slurry of cells in 1:1 mixture (v/v) of media and Matrigel (Corning). Xenografts were generally palpable within 3-4 weeks after injection. 4A06 was functionalized with desferrioxamine (DFO) and subsequently radiolabeled with Zr-89. Tumor-bearing mice received ˜300 μCi of ⁸⁹Zr-4A06 in 100 μL saline solution volume intravenously using a custom mouse tail vein catheter with a 28-gauge needle and a 100-150 mm long polyethylene microtubing. 4A06 was functionalized with DOTA and radiolabeled with Lu-177 as previously described (Moroz et al., Clin Cancer Res, 6(14):3608-15, 2020). Mice bearing subcutaneous UMUC3 tumors received ¹⁷⁷Lu-4A06 (400 μCi) or vehicle (saline) via tail vein at day 0 and day 7 of the study period. Mice were arranged in treatment arms using a simple randomization approach. Animals were weighed at the time of injection, and three times per week until the completion of the study. Tumor volume measurements were calculated at the same time points with calipers. The study endpoints were death due to tumor volume >2000 mm³ or ≥20% loss in mouse body weight. The researcher performing the tumor volume and body weight measurements was blinded to the treatment arms.

Small animal PET/CT. Mice were imaged on a small animal PET/CT scanner (Inveon, Siemens Healthcare, Malvern, PA). Animals were typically scanned for 30 minutes for PET, and the CT acquisition was performed for 10 minutes. The co-registration between PET and CT images was obtained using the rigid transformation matrix generated prior to the imaging data acquisition since the geometry between PET and CT remained constant for each of PET/CT scans using the combined PET/CT scanner. For microPET/CT data, PET images were reconstructed using the ordered subsets expectation maximization algorithm (OSEM) provided by the scanner manufacturer. The parameters for OSEM were 16 subsets and 4 iterations, and the resulting reconstructed image volume was in a matrix of 128×128×159 with a voxel size of 0.0776 mm×0.0775 mm×0.0796 mm. CT images for attenuation correction were reconstructed using a conebeam Feldkamp algorithm provided by the scanner manufacturer. The data were acquired using x-ray tube voltage of 80 kVp and current of 0.5 mA for 120 angular steps over 220 degrees, and 175 ms of exposure at each angular step. The reconstructed CT volume was in a matrix of 512×512×700 with a voxel size of 0.195 mm×0.195 mm×0.195 mm. The precalibrated scaling was used to convert the CT images to attenuation maps for correction in PET reconstruction. For SUV computation, we used freeware software, Amide (amide.sourceforge.net), and used its automated SUV calculation tool by entering decay-corrected injected activity and the animal weight. For each volume of interest, a spherical VOI (2-3 mm diameter) was drawn, and SUV was calculated by VOI statistics.

Biodistribution studies. At a dedicated time after radiotracer injection, animals were euthanized. Blood was harvested via cardiac puncture. Tissues were removed, weighed, and counted on a Hidex automatic gamma counter (Turku, Finland). The activity of the injected radiotracer was calculated and used to determine the total number of counts per minute by comparison with a standard of known activity. The data were background- and decay-corrected and expressed as the percentage of the injected dose/weight of the biospecimen in grams (% ID/g).

Statistical analysis. For the transcriptomic analysis, correlation was calculated by Spearman's rank correlation. ANOVA and Kruskal-Wallis tests were used to test for differences when there were more than two groups, and the Wilcoxon rank-sum test was used to test for differences between two groups unless otherwise stated. Binary comparisons between two treatment arms were made with an unpaired, two-tailed Student's t-test. Differences at the 95% confidence level (P<0.05) were considered statistically significant. Unless otherwise stated, all data were expressed as mean±standard deviation.

### Results

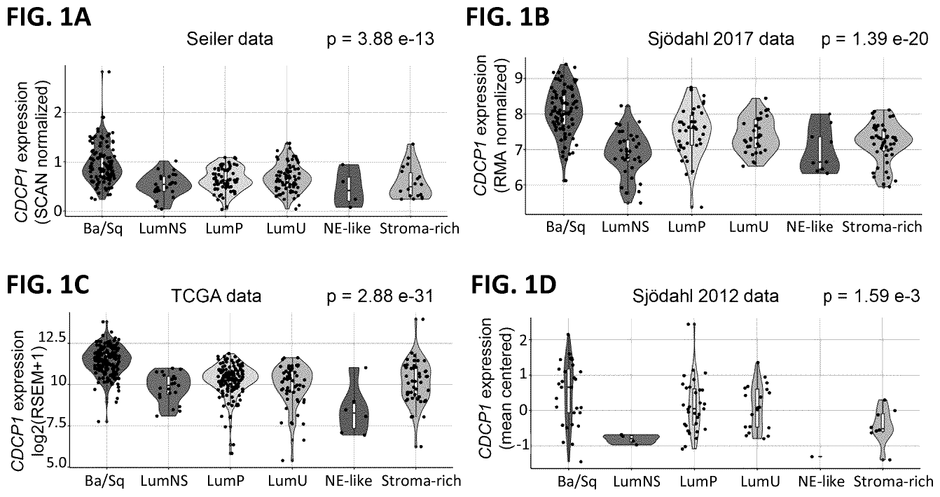

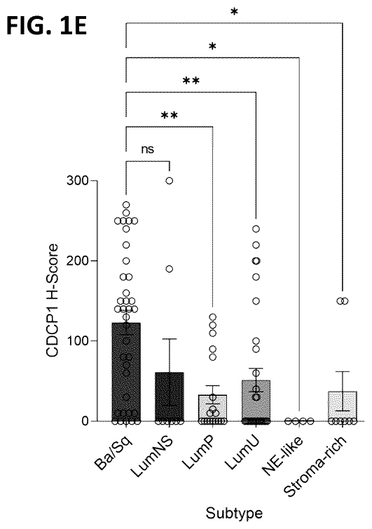

CDCP1 is expressed in all molecular subtypes of bladder cancer, with the highest expression in the basal/squamous subtype. To assess CDCP1 mRNA expression across various molecular subtypes, four cohorts of patients with localized, muscle-invasive bladder cancer (MIBC) were analyzed. Median CDCP1 expression was elevated in the basal/squamous (Ba/Sq) subtype of MIBC relative to the luminal (LumP, LumNS and LumU), stromal-rich and neuroendocrine (NE)-like subtypes (FIG. 1A-1D). To determine whether CDCP1 is enriched in patients with bladder cancer, median expression of CDCP1 in healthy patients was compared to expression of CDCP1 in a similarly normalized dataset of patients with MIBC. As demonstrated in FIG. 1E, immunohistochemistry (IHC) using a tissue microarray (TMA) shows CDCP1 protein was enriched in Ba/Sq tumors by immunohistochemistry (IHC). No significant associations between quartiles of CDCP1 expression and overall survival were seen in cohorts for which retrospective clinical data were available (see, Table 1-1).

TABLE 1-1
Pairwise comparisons of CDCP1 expression between BC consensus subtypes
Seiler dataset

| | |
|---|---|
| ANOVA p value: | 3.88E−13 |
| Kruskal-Wallis p value: | 4.49E−11 |

P values for pairwise Wilcoxon rank-sum test

| | Ba/Sq | LumNS | LumP | LumU | NE-like |
|---|---|---|---|---|---|
| LumNS | 4.23E−07 | NA | NA | NA | NA |
| LumP | 6.76E−08 | 0.05075339 | NA | NA | NA |
| LumU | 5.75E−07 | 0.03695089 | 0.6531837 | NA | NA |
| NE-like | 3.67E−02 | 0.77577534 | 0.3262516 | 0.2375863 | NA |
| Stroma-rich | 1.39E−04 | 0.94989805 | 0.1587395 | 0.1074993 | 0.4341763 |

Sjodahl 2017 dataset

| | |
|---|---|
| ANOVA p value: | 1.39E−20 |
| Kruskal-Wallis p value: | 5.24E−17 |

P values for pairwise Wilcoxon rank-sum test

| | Ba/Sq | LumNS | LumP | LumU | NE-like |
|---|---|---|---|---|---|
| LumNS | 4.76E−12 | NA | NA | NA | NA |
| LumP | 2.48E−05 | 0.00017671 | NA | NA | NA |
| LumU | 5.67E−07 | 0.00128949 | 0.35698733 | NA | NA |
| NE-like | 1.46E−04 | 0.94155928 | 0.03703647 | 0.06471567 | NA |
| Stroma-rich | 1.53E−11 | 0.02544874 | 0.02032813 | 0.21558074 | 0.3965704 |

TCGA dataset

| | |
|---|---|
| ANOVA p value: | 2.88E−31 |
| Kruskal-Wallis p value: | 7.93E−30 |

P values for pairwise Wilcoxon rank-sum test

| | Ba/Sq | LumNS | LumP | LumU | NE-like |
|---|---|---|---|---|---|
| LumNS | 6.36E−10 | NA | NA | NA | NA |
| LumP | 1.71E−22 | 0.01912351 | NA | NA | NA |
| LumU | 3.29E−15 | 0.31386544 | 0.14242979 | NA | NA |
| NE-like | 2.14E−04 | 0.03586365 | 0.00618171 | 0.02323164 | NA |
| Stroma-rich | 1.20E−10 | 0.249711 | 0.3841652 | 0.73220628 | 0.01069538 |

Sjodahl 2012 dataset

| | |
|---|---|
| ANOVA p value: | 0.00159194 |
| Kruskal-Wallis p value: | 0.00121189 |

P values for pairwise Wilcoxon rank-sum test

| | Ba/Sq | LumNS | LumP | LumU | NE-like |
|---|---|---|---|---|---|
| LumNS | 0.00459433 | NA | NA | NA | NA |
| LumP | 0.0540352 | 0.00359053 | NA | NA | NA |
| LumU | 0.06229299 | 0.00225861 | 0.91629071 | NA | NA |
| NE-like | 0.13333333 | 0.4 | 0.0625 | 0.0952381 | NA |
| Stroma-rich | 0.00495674 | 0.04848485 | 0.04798674 | 0.06251076 | 0.4444444 |

CDCP1 is expressed in TROP2 and NECTIN4-low and null bladder cancer.

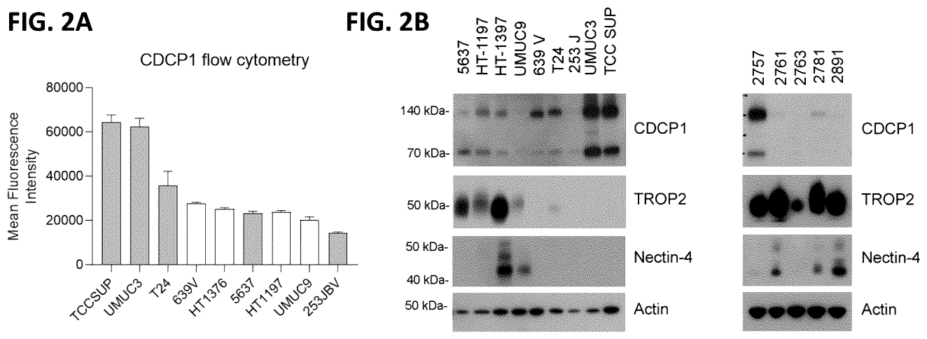

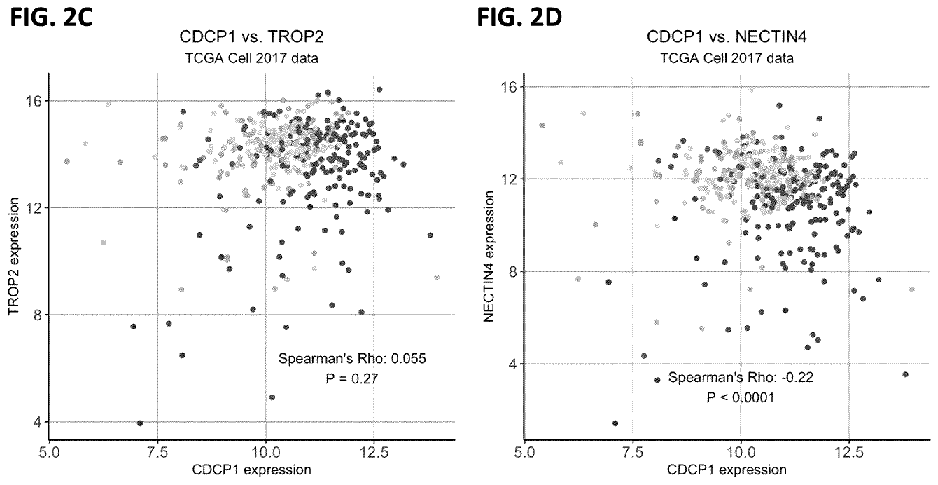

CDCP1 protein expression in human bladder cancer cell lines representing luminal (5637, HT-1197, HT-1397, UMUC9) and basal subtypes (639V, T24, 253JBV, UMUC3, TCCSUP) was evaluated. Cell surface protein expression was observed in all cell lines by flow cytometry using the 4A06 antibody (FIG. 2A). UMUC3 and TCC SUP had the highest expression of CDCP1, while UMUC9 and 253 JBV had the lowest relative level. Cell surface receptor numbers were further quantified using ¹²⁵I-labeled 4A06 antibody. Saturation binding assays showed that UMUC3 and TCC SUP had ˜1×10⁶ receptors per cell, while UMUC9 and 5637 had ˜1×10⁵ receptors per cell (see, Table 1-2).

TABLE 1-2
CDCP1 receptor density on human bladder cancer cell lines using ¹²⁵I-4A06

| | | Bmax | | Kd | | | Receptors/ |
|---|---|---|---|---|---|---|---|
| Cell Line | Histology | (fmol/mg) | 95% CI | (nM) | 95% CI | R² | cell |
| UMUC3 | Basal | 27,301 | 22,918-32,291 | 1.84 | 1.0-3.2 | 0.95 | 1.49 × 10⁶ |
| TCC SUP | Basal | 23,784 | 20,987-26,796 | 0.583 | 0.33-0.96 | 0.9 | 1.29 × 10⁶ |
| 5637 | Basal | 10,037 | 9,390-10,711 | 0.284 | 0.19-0.39 | 0.94 | 5.48 × 10⁵ |
| UMUC9 | Luminal | 3,961 | 3,253-4,860 | 0.181 | 0.056-0.45 | 0.88 | 2.16 × 10⁵ |

Full length CDCP1 (˜140 kDa) can be proteolytically cleaved on the cell surface to generate a truncated form (˜90 kDa). Some recent studies have suggested either form promotes cancer aggressiveness through discrete mechanisms. On this basis, CDCP1 expression was evaluated using immunoblot. Full length and cleaved CDCP1 were detected in cell lines (FIG. 2B). In general, full length CDCP1 was equivalently or more abundantly expressed than cleaved CDCP1. In one cell line, 5637, cleaved CDCP1 was more abundant than the full-length form. CDCP1 was robustly expressed in UMUC3, T24, 639V and TCCSUP lines, which are all negative for NECTIN4 and TROP2. In addition, CDCP1 was detected in three of five bladder cancer patient-derived xenografts (PDX) (FIG. 2B). CDCP1 expression was either not correlated or inversely correlated with both TROP2 and NECTIN4 expression (FIG. 2C-2D).

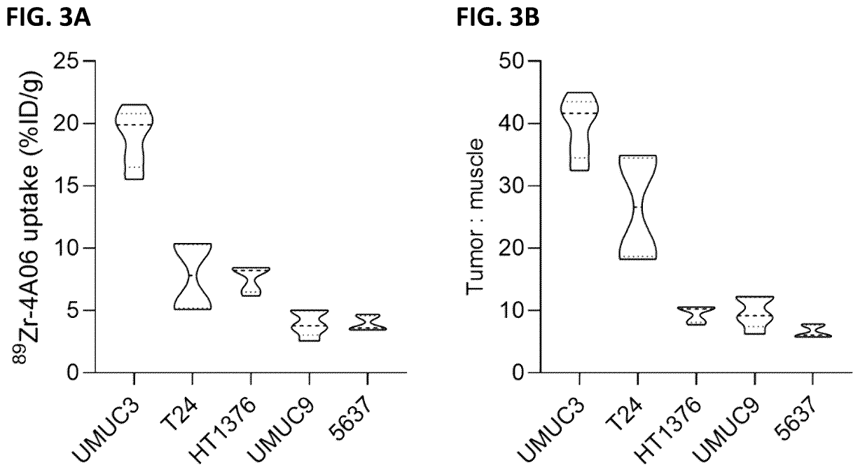

CDCP1 can be targeted on bladder cancer tumors for nuclear imaging and therapy with radiolabeled antibodies. To understand if CDCP1 can be targeted for bladder cancer therapy, the tumoral uptake of the 4A06 antibody was assessed. The 4A06 antibody was functionalized with desferrioxamine and radiolabeled with Zr-89. PET and biodistribution studies were conducted at 72 hours post injection of ⁸⁹Zr-4A06, which shows the peak tumor to background values at this time point (Moroz et al., Clin Cancer Res, 6(14):3608-15, 2020). Five human xenograft models were used, representing higher (UMUC3, T24, HT1376) and relatively lower (UMUC9, 5637) CDCP1 expression in vitro. Both on PET and ex vivo biodistribution, relative levels of ⁸⁹Zr-4A06 uptake in tumors aligned with the relative CDCP1 expression level observed in vitro (FIG. 3A). The highest tumoral uptake of ⁸⁹Zr-4A06 was observed in UMUC3 tumors at ˜20% ID/g. In all cases, the tumor to background ratio was >1 (FIG. 3B).

Whether CDCP1-directed targeted radiotherapy (TRT) could be applied to treat bladder cancer tumors was subsequently tested. Recent U.S. Food & Drug Administration approvals of LUTATHERA® (Lutetium Lu-177 Dotate, marketed by Novartis AG) for neuroendocrine tumors, AZEDRA® (Iobenguane I-131, marketed by Progenics Pharmaceuticals, Inc.) for pediatric malignancies, and PLUVICTOR (Lutetium Lu-177 Vipivotide Tetraxetan, marketed by Novartis AG) for prostate cancer underscore that TRT can be effective against biologically diverse metastatic solid tumor types (Strosberg et al., N Engl J Med, 376(2):125-35, 2017; Sartor et al., N Engl J Med, 385(12):1091-103, 2021; and Hofman et al., Lancet, 397(10276):797-804, 2021). However, none of LUTATHERA®, AZEDRA® and PLUVICTO® are antibody-based therapeutic modalities.

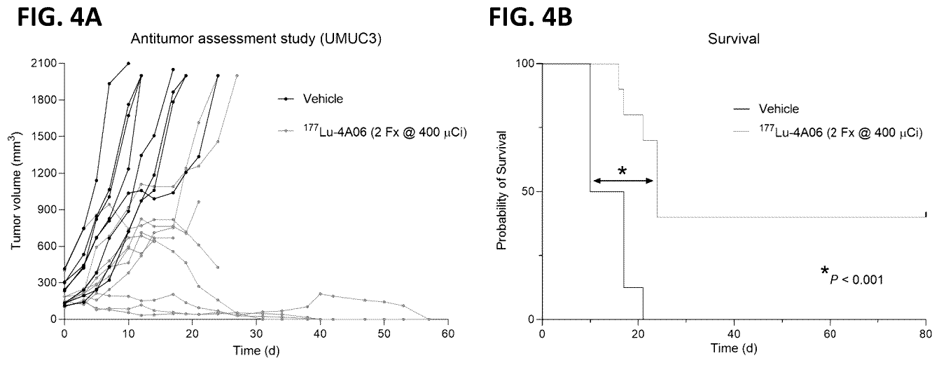

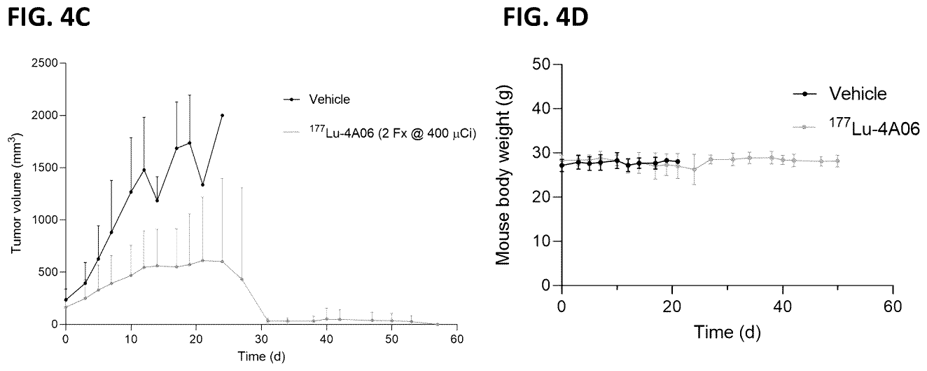

To test the antitumor effects of CDCP1-TRT, 4A06 IgG1 was first conjugated to the chelator 1,4,7,10-tetraazacyclododecane-1,4,7,10-tetraacetic acid (DOTA). DOTA-4A06 was radiolabeled with Lu-177. ¹⁷⁷Lu-4A06 was administered intravenously in two doses (400 μCi/dose) on day 0 and day 5 of the study period to mice bearing subcutaneous UMUC3 tumors. Treatment with ¹⁷⁷Lu-4A06 significantly suppressed tumor growth compared to vehicle controls (FIG. 4A and 4C). Notably, four of nine tumors in the treated arm underwent a complete response with no tumor outgrowth even at 120 days post injection. Assessing the entire cohort, ¹⁷⁷Lu-4A06 significantly extended survival compared to the control arm. As shown in FIG. 4B, mice receiving vehicle had a median survival of 13 days, while mice receiving ¹⁷⁷Lu-4A06 had a median survival of 24 days (Hazard ratio=4, P<0.001).

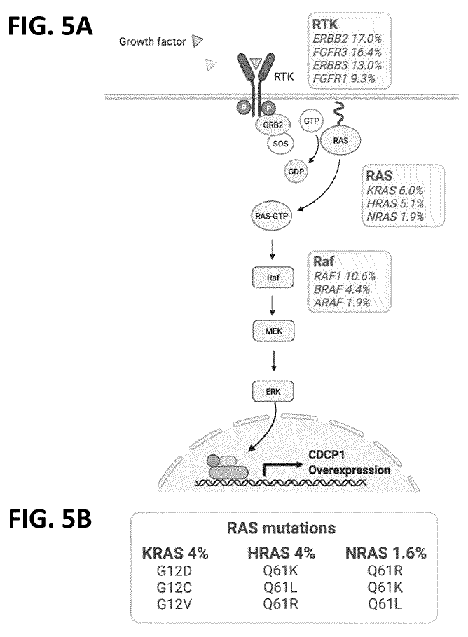

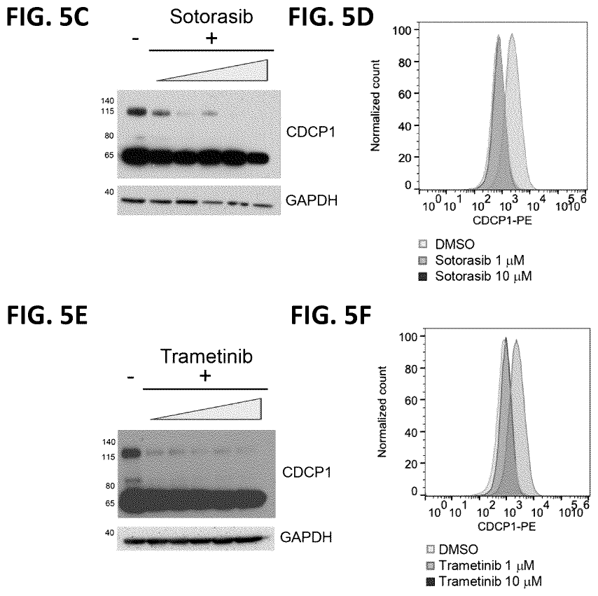

Hyperactive RAS signaling promotes CDCP1 overexpression in bladder cancer cells. An exploratory study was carried out to probe the mechanisms driving CDCP1 overexpression in BC. RAS/Raf/MEK signaling has been previously shown to induce expression of CDCP1 in other cancers (FIG. 5A) (Uekita et al., Mol Cancer Res, 12(10):1449-59, 2014). Hyperactive RAS signaling is a relatively common feature of BC due to mutations in RAS isoforms, upstream RAS inducers, and downstream effectors (FIG. 5B). Whether the RAS/Raf/MEK signaling pathway regulates CDPC1 in BC was then tested. Treatment of UMUC3 cells (which harbor a KRAS G12C mutation) with G12C inhibitor sotorasib for 48 hours suppressed CDCP1 expression in a dose dependent fashion on immunoblot and flow cytometry (FIG. 5C-5D). Treatment of UMUC3 cells with the MEK inhibitor trametinib for 48 hours also suppressed CDCP1 expression in a dose dependent fashion on immunoblot and flow cytometry (FIGS. 5E-5F). These data reveal a role for CDCP1-directed therapeutics in mutant RAS bladder cancers.

### Conclusions

Profiling mRNA and protein expression in BC biopsies showed that CDCP1 is expressed in all of the consensus subtypes, with the highest levels found in the aggressive basal/squamous subtype and lowest levels in the NE-like subtype. Flow cytometry and saturation binding studies confirmed that CDCP1 is expressed on the cell surface of both basal and luminal human bladder cancer cell lines. Saturation binding studies in cell line models showed a range of overexpression from 10⁵−10⁶ receptors per cell. Immunoblot of human BC cell lines and PDX samples demonstrated that both full length and cleaved CDCP1 are expressed.

Interestingly, robust CDCP1 expression was found in several NECTIN4 and TROP2 negative BC lines, indicating that CDCP1-directed therapies may benefit a different molecular subset of patients. PET/CT studies with ⁸⁹Zr-4A06 detected tumor autonomous expression of CDCP1 in four human bladder cancer models of either basal or luminal histology. An antitumor assessment study showed that ¹⁷⁷Lu-4A06 potently suppressed the growth of UMUC3 tumors compared to vehicle controls and extended overall survival. Of note, a durable complete response was observed in 44% of the mice (four of nine) in the ¹⁷⁷Lu-4A06 treated cohort.

Our results showing potent inhibition of an aggressive BC xenograft model support revisiting TRT-directed therapies for localized and advanced BC.

We claim:

1. A method for treating CUB domain-containing protein 1 (CDCP1)-positive bladder cancer, comprising:
administering to a subject in need thereof a therapeutic radioisotope-labeled antibody or antibody fragment that specifically binds CDCP1 ectodomain to treat the bladder cancer, wherein the therapeutic radioisotope is an alpha-emitter and/or a beta-minus-emitter.

2. The method of claim 1, wherein the antibody or antibody fragment comprises a light chain CDR1 comprising SEQ ID NO:11, a light chain CDR2 comprising SEQ ID NO:13, and:
(a) a light chain CDR3 comprising SEQ ID NO:20, a heavy chain CDR1 comprising SEQ ID NO:17, a heavy chain CDR2 comprising SEQ ID NO:18, and a heavy chain CDR3 comprising SEQ ID NO:19;
(b) a light chain CDR3 comprising SEQ ID NO:24, a heavy chain CDR1 comprising SEQ ID NO:21, a heavy chain CDR2 comprising SEQ ID NO:22, and a heavy chain CDR3 comprising SEQ ID NO:23;
(c) a light chain CDR3 comprising SEQ ID NO:28, a heavy chain CDR1 comprising SEQ ID NO:25, a heavy chain CDR2 comprising SEQ ID NO:26, and a heavy chain CDR3 comprising SEQ ID NO:27;
(d) a light chain CDR3 comprising SEQ ID NO:32, a heavy chain CDR1 comprising SEQ ID NO:29, a heavy chain CDR2 comprising SEQ ID NO:30, and a heavy chain CDR3 comprising SEQ ID NO:31;
(e) a light chain CDR3 comprising SEQ ID NO:36, a heavy chain CDR1 comprising SEQ ID NO:33, a heavy chain CDR2 comprising SEQ ID NO:34, and a heavy chain CDR3 comprising SEQ ID NO:35;
(f) a light chain CDR3 comprising SEQ ID NO:40, a heavy chain CDR1 comprising SEQ ID NO:37, a heavy chain CDR2 comprising SEQ ID NO:38, and a heavy chain CDR3 comprising SEQ ID NO:39;
(g) a light chain CDR3 comprising SEQ ID NO:44, a heavy chain CDR1 comprising SEQ ID NO:41, a heavy chain CDR2 comprising SEQ ID NO:42, and a heavy chain CDR3 comprising SEQ ID NO:43;
(h) a light chain CDR3 comprising SEQ ID NO:48, a heavy chain CDR1 comprising SEQ ID NO:45, a heavy chain CDR2 comprising SEQ ID NO:46, and a heavy chain CDR3 comprising SEQ ID NO:47;
(i) a light chain CDR3 comprising SEQ ID NO:52, a heavy chain CDR1 comprising SEQ ID NO: 49, a heavy chain CDR2 comprising SEQ ID NO:50, and a heavy chain CDR3 comprising SEQ ID NO:51; or
(j) a light chain CDR3 comprising SEQ ID NO:56, a heavy chain CDR1 comprising SEQ ID NO:53, a heavy chain CDR2 comprising SEQ ID NO:54, and a heavy chain CDR3 comprising SEQ ID NO:55.

3. The method of claim 1, wherein the antibody or antibody fragment comprises light chain CDRs of SEQ ID NO:11, SEQ ID NO:13 and SEQ ID NO:20, and heavy chain CDRs of SEQ ID NO:17, SEQ ID NO:18, and SEQ ID NO:19.

4. The method of claim 1, wherein the antibody or antibody fragment comprises a heavy chain variable domain (VH) and a light chain variable domain (VL), and the VH comprises an amino acid sequence having at least 95% sequence identity to SEQ ID NO:57, and the VL comprises an amino acid sequence having at least 95% sequence identity to SEQ ID NO:58.

5. The method of claim 1, wherein the antibody or antibody fragment comprises a heavy chain variable domain (VH) and a light chain variable domain (VL), and the VH comprises the amino acid sequence of SEQ ID NO:57, and the VL comprises the amino acid sequence of SEQ ID NO:58.

6. The method of claim 1, wherein the antibody or antibody fragment is a recombinant IgG₁ antibody.

7. The method of claim 1, wherein the therapeutic radioisotope is selected from the group consisting of ⁶⁷Cu, ⁹⁰Y, ¹³¹I, ¹⁵³Sm, ¹⁷⁷Lu, ²¹¹At, ²¹²Pb, ²²³Ra, ²²⁵Ac, and ²²⁷Th.

8. The method of claim 1, wherein the therapeutic radioisotope is ¹⁷⁷Lu.

9. The method of claim 1, wherein the bladder cancer is muscle-invasive bladder cancer (MIBC).

10. The method of claim 9, wherein the bladder cancer is a basal/squamous (Ba/Sq) subtype of MIBC.

11. The method of claim 1, wherein cells of the bladder cancer are TROP2-negative and/or NECTIN4-negative.

12. The method of claim 1, further comprising prior to administering the therapeutic radioisotope-labeled antibody or antibody fragment:
determining the bladder cancer is CDCP1-positive by immunohistochemistry analysis of a bladder cancer biopsy obtained from the subject.

13. The method of claim 1, further comprising prior to administering the therapeutic radioisotope-labeled antibody or antibody fragment:
determining the bladder cancer is CDCP1-positive by radionuclide imaging following administering a diagnostic radioisotope-labeled antibody or antibody fragment that specifically binds the CDCP1 ectodomain to the subject, wherein the diagnostic radioisotope is a positron-emitter.

14. The method of claim 13, wherein the diagnostic radioisotope is selected from the group consisting of ¹⁸F, ⁴⁴Sc ⁶⁴Cu, ⁶⁸Ga, ⁸⁹Zr, and ¹²⁴I.

15. The method of claim 13, wherein the diagnostic radioisotope is ⁸⁹Zr.

16. The method of claim 13, wherein the radionuclide imagining comprises positron emission tomography (PET) or single-photon emission computed tomography (SPECT).

17. The method of claim 1, wherein one or both of the therapeutic radioisotope-labeled antibody or antibody fragment and the diagnostic radioisotope-labeled antibody or fragment are administered intravenously.

18. The method of claim 1, wherein one or both of the therapeutic radioisotope-labeled antibody or antibody fragment and the diagnostic radioisotope-labeled antibody or fragment are administered by intravesicular deliver to the bladder.

19. A method for assessing CUB domain-containing protein 1 (CDCP1) expression, comprising:
(a) administering a diagnostic radioisotope-labeled antibody or antibody fragment that specifically binds the CDCP1 ectodomain to a subject having bladder cancer; and
(b) visualizing binding of the antibody or antibody fragment to cells of the subject by radionuclide imaging to assess CDC1 expression, wherein
the diagnostic radioisotope is a positron-emitter.

20. The method of claim 19, wherein the antibody or antibody fragment comprises a light chain CDR1 comprising SEQ ID NO:11, a light chain CDR2 comprising SEQ ID NO:13, and:
(a) a light chain CDR3 comprising SEQ ID NO:20, a heavy chain CDR1 comprising SEQ ID NO:17, a heavy chain CDR2 comprising SEQ ID NO:18, and a heavy chain CDR3 comprising SEQ ID NO:19;
(b) a light chain CDR3 comprising SEQ ID NO:24, a heavy chain CDR1 comprising SEQ ID NO:21, a heavy chain CDR2 comprising SEQ ID NO:22, and a heavy chain CDR3 comprising SEQ ID NO:23;
(c) a light chain CDR3 comprising SEQ ID NO:28, a heavy chain CDR1 comprising SEQ ID NO:25, a heavy chain CDR2 comprising SEQ ID NO:26, and a heavy chain CDR3 comprising SEQ ID NO:27;
(d) a light chain CDR3 comprising SEQ ID NO:32, a heavy chain CDR1 comprising SEQ ID NO:29, a heavy chain CDR2 comprising SEQ ID NO:30, and a heavy chain CDR3 comprising SEQ ID NO:31;
(e) a light chain CDR3 comprising SEQ ID NO:36, a heavy chain CDR1 comprising SEQ ID NO:33, a heavy chain CDR2 comprising SEQ ID NO:34, and a heavy chain CDR3 comprising SEQ ID NO:35;
(f) a light chain CDR3 comprising SEQ ID NO:40, a heavy chain CDR1 comprising SEQ ID NO:37, a heavy chain CDR2 comprising SEQ ID NO:38, and a heavy chain CDR3 comprising SEQ ID NO:39;
(g) a light chain CDR3 comprising SEQ ID NO:44, a heavy chain CDR1 comprising SEQ ID NO:41, a heavy chain CDR2 comprising SEQ ID NO:42, and a heavy chain CDR3 comprising SEQ ID NO:43;
(h) a light chain CDR3 comprising SEQ ID NO:48, a heavy chain CDR1 comprising SEQ ID NO:45, a heavy chain CDR2 comprising SEQ ID NO:46, and a heavy chain CDR3 comprising SEQ ID NO:47;
(i) a light chain CDR3 comprising SEQ ID NO:52, a heavy chain CDR1 comprising SEQ ID NO:49, a heavy chain CDR2 comprising SEQ ID NO:50, and a heavy chain CDR3 comprising SEQ ID NO:51; or
(j) a light chain CDR3 comprising SEQ ID NO:56, a heavy chain CDR1 comprising SEQ ID NO:53, a heavy chain CDR2 comprising SEQ ID NO:54, and a heavy chain CDR3 comprising SEQ ID NO:55.

21. The method of claim 19, wherein the antibody or antibody fragment comprises light chain CDRs of SEQ ID NO:11, SEQ ID NO:13 and SEQ ID NO:20, and heavy chain CDRs of SEQ ID NO:17, SEQ ID NO:18, and SEQ ID NO:19.

22. The method of claim 19, wherein the antibody or antibody fragment comprises a heavy chain variable domain (VH) and a light chain variable domain (VL), and the VH comprises an amino acid sequence having at least 95% sequence identity to SEQ ID NO:57, and the VL comprises an amino acid sequence having at least 95% sequence identity to SEQ ID NO:58.

23. The method of claim 19, wherein the antibody or antibody fragment comprises a heavy chain variable domain (VH) and a light chain variable domain (VL), and the VH comprises the amino acid sequence of SEQ ID NO:57, and the VL comprises the amino acid sequence of SEQ ID NO:58.

24. The method of claim 19, wherein the diagnostic radioisotope is selected from the group consisting of ¹⁸F, ⁴⁴Sc ⁶⁴Cu, ⁶⁸Ga, ⁸⁹Zr, and ¹²⁴I.

25. The method of claim 19, wherein the diagnostic radioisotope is ⁸⁹Zr.

26. The method of claim 19, wherein the radionuclide imagining comprises positron emission tomography (PET) or single-photon emission computed tomography (SPECT).

27. The method of claim 19, wherein the bladder cancer is muscle-invasive bladder cancer (MIBC).

28. The method of claim 27, wherein the bladder cancer is a basal/squamous (Ba/Sq) subtype of MIBC.

29. The method of claim 19, wherein cells of the bladder cancer are TROP2-negative and/or NECTIN4-negative.

30. The method of claim 19, further comprising determining the subject has a CUB domain-containing protein 1 (CDCP1)-positive bladder cancer, and treating the CDCP1-positive bladder cancer by a method comprising:
administering to the subject a therapeutic radioisotope-labeled antibody or antibody fragment that specifically binds CDCP1 ectodomain to treat the bladder cancer, wherein the therapeutic radioisotope is an alpha-emitter and/or a beta-minus-emitter.


SEQUENCE LISTING (80 sequences)

SEQ ID NO: 1 - protein, 246 aa, synthetic construct
EISEVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSHHHHHH

SEQ ID NO: 2 - protein, 31 aa, synthetic construct
EISEVQLVESGGGLVQPGGSLRLSCAASGFN

SEQ ID NO: 3 - protein, 6 aa, synthetic construct
FSSSSI

SEQ ID NO: 4 - protein, 15 aa, synthetic construct
HWVRQAPGKGLEWVA

SEQ ID NO: 5 - protein, 10 aa, synthetic construct
SISSSYGYTY

SEQ ID NO: 6 - protein, 39 aa, synthetic construct
YADSVKGRFTISADTSKNTAYLQMNSLRAEDTAVYYCAR

SEQ ID NO: 7 - protein, 15 aa, synthetic construct
TVRGSKKPYFSGWAM

SEQ ID NO: 8 - protein, 13 aa, synthetic construct
DYWGQGTLVTVSS

SEQ ID NO: 9 - protein, 226 aa, synthetic construct
SDIQMTQSPSSLSASVGDRVTITCRASQSVSSAVAWYQQKPGKAPKLLIYSASSLYSGVPSRFSGSRSGTDFTLTISSLQPEDFATYYCQQSSYSLITFGQGTKVEIKRTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGECGGSDYKDDDDK

SEQ ID NO: 10 - protein, 24 aa, synthetic construct
SDIQMTQSPSSLSASVGDRVTITC

SEQ ID NO: 11 - protein, 11 aa, synthetic construct
RASQSVSSAVA

SEQ ID NO: 12 - protein, 15 aa, synthetic construct
WYQQKPGKAPKLLIY

SEQ ID NO: 13 - protein, 7 aa, synthetic construct
SASSLYS

SEQ ID NO: 14 - protein, 34 aa, synthetic construct
GVPSRFSGSRSGTDFTLTISSLQPEDFATYYCQQ

SEQ ID NO: 15 - protein, 6 aa, synthetic construct
SSYSLI

SEQ ID NO: 16 - protein, 11 aa, synthetic construct
TFGQGTKVEIK

SEQ ID NO: 17 - protein, 6 aa, synthetic construct
LSYYYI

SEQ ID NO: 18 - protein, 10 aa, synthetic construct
SIYSSSSYTS

SEQ ID NO: 19 - protein, 5 aa, synthetic construct
AYYGF

SEQ ID NO: 20 - protein, 7 aa, synthetic construct
SYYYYPI

SEQ ID NO: 21 - protein, 6 aa, synthetic construct
ISYYSM

SEQ ID NO: 22 - protein, 10 aa, synthetic construct
SISPYSGYTS

SEQ ID NO: 23 - protein, 5 aa, synthetic construct
GYYAL

SEQ ID NO: 24 - protein, 7 aa, synthetic construct
YYYFYPF

SEQ ID NO: 25 - protein, 6 aa, synthetic construct
ISYYYM

SEQ ID NO: 26 - protein, 10 aa, synthetic construct
SIYSSYGYTS

SEQ ID NO: 27 - protein, 5 aa, synthetic construct
VYYGF

SEQ ID NO: 28 - protein, 7 aa, synthetic construct
SYYVYPI

SEQ ID NO: 29 - protein, 6 aa, synthetic construct
ISYYYI

SEQ ID NO: 30 - protein, 10 aa, synthetic construct
SIYPYYGSTY

SEQ ID NO: 31 - protein, 5 aa, synthetic construct
AYYGF

SEQ ID NO: 32 - protein, 7 aa, synthetic construct
SYWSFPI

SEQ ID NO: 33 - protein, 6 aa, synthetic construct
LYYSYM

SEQ ID NO: 34 - protein, 10 aa, synthetic construct
YISPYSGSTY

SEQ ID NO: 35 - protein, 6 aa, synthetic construct
YSYSAL

SEQ ID NO: 36 - protein, 8 aa, synthetic construct
SSWHYHLF

SEQ ID NO: 37 - protein, 6 aa, synthetic construct
IYSYYI

SEQ ID NO: 38 - protein, 10 aa, synthetic construct
SIYPYYGYTS

SEQ ID NO: 39 - protein, 5 aa, synthetic construct
AYYGM

SEQ ID NO: 40 - protein, 7 aa, synthetic construct
SYFYWPI

SEQ ID NO: 41 - protein, 6 aa, synthetic construct
ISSYYM

SEQ ID NO: 42 - protein, 10 aa, synthetic construct
SIYPYSGYTY

SEQ ID NO: 43 - protein, 5 aa, synthetic construct
AYYAM

SEQ ID NO: 44 - protein, 7 aa, synthetic construct
SYYVYPI

SEQ ID NO: 45 - protein, 6 aa, synthetic construct
LYSYYI

SEQ ID NO: 46 - protein, 10 aa, synthetic construct
SIYPYYSSTS

SEQ ID NO: 47 - protein, 5 aa, synthetic construct
YYYAM

SEQ ID NO: 48 - protein, 9 aa, synthetic construct
GYAGSWHPI

SEQ ID NO: 49 - protein, 6 aa, synthetic construct
IYSYSM

SEQ ID NO: 50 - protein, 10 aa, synthetic construct
SISPYYSYTS

SEQ ID NO: 51 - protein, 5 aa, synthetic construct
AYYAL

SEQ ID NO: 52 - protein, 7 aa, synthetic construct
SYWYYPI

SEQ ID NO: 53 - protein, 6 aa, synthetic construct
ISYYYM

SEQ ID NO: 54 - protein, 10 aa, synthetic construct
SIYSSSSYTS

SEQ ID NO: 55 - protein, 5 aa, synthetic construct
SYYAM

SEQ ID NO: 56 - protein, 7 aa, synthetic construct
SYYVYPI

SEQ ID NO: 57 - protein, 116 aa, synthetic construct
EVQLVESGGGLVQPGGSLRLSCAASGFNLSYYYIHWVRQAPGKGLEWVASIYSSSSYTSYADSVKGRFTISADTSKNTAYLQMNSLRAEDTAVYYCARAYYGFDYWGQGTLVTVSS

SEQ ID NO: 58 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYYYYPITFGQGTKVEIK

SEQ ID NO: 59 - protein, 116 aa, synthetic construct
EVQLVESGGGLVQPGGSLRLSCAASGFNISYYSMHWVRQAPGKGLEWVASISPYSGYTSYADSVKGRFTISADTSKNTAYLQMNSLRAEDTAVYYCARGYYALDYWGQGTLVTVSS

SEQ ID NO: 60 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYYYFYPFTFGQGTKVEIK

SEQ ID NO: 61 - protein, 116 aa, synthetic construct
EVQLVESGGGLVQPGGSLRLSCAASGFNISYYYMHWVRQAPGKGLEWVASIYSSYGYTSYADSVKGRFTISADTSKNTAYLQMNSLRAEDTAVYYCARVYYGFDYWGQGTLVTVSS

SEQ ID NO: 62 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYYVYPITFGQGTKVEIK

SEQ ID NO: 63 - protein, 116 aa, synthetic construct
EVQLVESGGGLVQPGGSLRLSCAASGFNISYYYIHWVRQAPGKGLEWVASIYPYYGSTYYADSVKGRFTISADTSKNTAYLQMNSLRAEDTAVYYCARAYYGFDYWGQGTLVTVSS

SEQ ID NO: 64 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYWSFPITFGQGTKVEIK

SEQ ID NO: 65 - protein, 117 aa, synthetic construct
EVQLVESGGGLVQPGGSLRLSCAASGFNLYYSYMHWVRQAPGKGLEWVAYISPYSGSTYYADSVKGRFTISADTSKNTAYLQMNSLRAEDTAVYYCARYSYSALDYWGQGTLVTVSS

SEQ ID NO: 66 - protein, 109 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSWHYHLFTFGQGTKVEIK

SEQ ID NO: 67 - protein, 116 aa, synthetic construct
EVQLVESGGGLVQPGGSLRLSCAASGFNIYSYYIHWVRQAPGKGLEWVASIYPYYGYTSYADSVKGRFTISADTSKNTAYLQMNSLRAEDTAVYYCARAYYGMDYWGQGTLVTVSS

SEQ ID NO: 68 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYFYWPITFGQGTKVEIK

SEQ ID NO: 69 - protein, 116 aa, synthetic construct
EVQLVESGGGLVQPGGSLRLSCAASGFNISSYYMHWVRQAPGKGLEWVASIYPYSGYTYYADSVKGRFTISADTSKNTAYLQMNSLRAEDTAVYYCARAYYAMDYWGQGTLVTVSS

SEQ ID NO: 70 - protein, 108 aa, synthetic construct; identical to SEQ ID NO: 62

SEQ ID NO: 71 - protein, 116 aa, synthetic construct
EVQLVESGGGLVQPGGSLRLSCAASGFNLYSYYIHWVRQAPGKGLEWVASIYPYYSSTSYADSVKGRFTISADTSKNTAYLQMNSLRAEDTAVYYCARYYYAMDYWGQGTLVTVSS

SEQ ID NO: 72 - protein, 110 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQGYAGSWHPITFGQGTKVEIK

SEQ ID NO: 73 - protein, 116 aa, synthetic construct
EVQLVESGGGLVQPGGSLRLSCAASGFNIYSYSMHWVRQAPGKGLEWVASISPYYSYTSYADSVKGRFTISADTSKNTAYLQMNSLRAEDTAVYYCARAYYALDYWGQGTLVTVSS

SEQ ID NO: 74 - protein, 108 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSYWYYPITFGQGTKVEIK

SEQ ID NO: 75 - protein, 116 aa, synthetic construct
EVQLVESGGGLVQPGGSLRLSCAASGFNISYYYMHWVRQAPGKGLEWVASIYSSSSYTSYADSVKGRFTISADTSKNTAYLQMNSLRAEDTAVYYCARSYYAMDYWGQGTLVTVSS

SEQ ID NO: 76 - protein, 108 aa, synthetic construct; identical to SEQ ID NO: 62

SEQ ID NO: 77 - protein, 126 aa, synthetic construct
EVQLVESGGGLVQPGGSLRLSCAASGFNFSSSSIHWVRQAPGKGLEWVASISSSYGYTYYADSVKGRFTISADTSKNTAYLQMNSLRAEDTAVYYCARTVRGSKKPYFSGWAMDYWGQGTLVTVSS

SEQ ID NO: 78 - protein, 107 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQSSYSLITFGQGTKVEIK

SEQ ID NO: 79 - protein, 14 aa, synthetic construct
DKTHTGGSHHHHHH

SEQ ID NO: 80 - protein, 11 aa, synthetic construct
GGSDYKDDDDK